(19) United States
(12) Patent Application Publication
(10) Pub. No.: US 2020/0283745 A1
(43) Pub. Date: Sep. 10, 2020

(54) NUCLEAR-TARGETED DNA REPAIR ENZYMES AND METHODS OF USE

(71) Applicant: Oregon Health & Science University, Portland, OR (US)

(72) Inventors: R. Stephen Lloyd, Portland, OR (US); Amanda K. McCullough, Portland, OR (US)

(73) Assignee: Oregon Health & Science University, Portland, OR (US)

(21) Appl. No.: 16/762,852

(22) PCT Filed: Nov. 14, 2018
(86) PCT No.: PCT/US2018/061108

§ 371 Date: May 8, 2020

Related U.S. Application Data
(60) Provisional application No. 62/585,947, filed on Nov. 14, 2017.

(51) Int. Cl. C12N 9/22; C07K 14/39; C12N 11/02; A61K 9/00; A61K 47/69; A61P 35/00

(52) U.S. Cl. CPC C12N 9/22; C07K 14/39; C12N 11/02; A61K 9/0014; A61K 47/6911; A61K 38/00; A61K 47/6903; C07K 2319/09; C07K 2319/10; C07K 2319/50; C07K 2319/21; A61P 35/00

(57) ABSTRACT

The present disclosure provides polypeptides that have the ability to repair DNA damage by recognizing and removing a wide variety of DNA damage and distortions in double-stranded DNA. In particular, the polypeptides have the ability to remove cyclobutane pyrimidine dimers (CPDs) and/or (6-4) photoproducts from DNA. The polypeptides include at least one heterologous targeting sequence.

### CROSS-REFERENCE TO RELATED APPLICATIONS

This is the U.S. National Phase of PCT/US2018/061108, filed Nov. 14, 2018, which claims priority to U.S. Provisional Application No. 62/585,947 filed on Nov. 14, 2017, and which is incorporated herein by reference in its entirety as if fully set forth herein.

### FIELD OF THE DISCLOSURE

The present disclosure provides polypeptides that have the ability to repair DNA damage. In particular, the polypeptides have the ability to remove cyclobutane pyrimidine dimers (CPDs) and/or (6-4) photoproducts from DNA. The polypeptides include at least one heterologous targeting sequence. The disclosure further provides polynucleotides encoding the polypeptides. Also provided by the disclosure are methods of using the polypeptides.

### BACKGROUND OF THE DISCLOSURE

Skin cancer is the uncontrolled growth of abnormal skin cells. It occurs when unrepaired DNA damage to skin cells (most often caused by ultraviolet radiation from sunshine or tanning beds) triggers mutations, or genetic defects, that lead the skin cells to multiply rapidly and form malignant tumors.

Non-melanoma skin cancers (NMSCs), including basal cell carcinoma (BCC) and squamous cell carcinoma (SCC) are the most prevalent types of human cancers, affecting over five million people in the United States annually, and costing billions of dollars for health care and loss of work (Guy et al., Am J Prey Med 43, 537-545, 2012; Wu et al., Future Oncol 11, 2967-2974, 2015; Bickers et al., J Am Acad Dermatol 55, 490-500, 2006). Non-melanoma skin cancers are almost exclusively restricted to portions of the body that are most frequently exposed to sunlight. The underlying mechanism is that DNA is damaged by the UVB and UVA spectra of sunlight and if these DNA photoproducts are not repaired, subsequent rounds of replication result in permanent genetic alterations (mutations). These mutations are the precursors to genetic alterations that can eventually lead to cancer. All mammals have only one DNA repair system, the nucleotide excision repair (NER) pathway, to remove the major forms of UV-induced DNA damage (cyclobutane pyrimidine dimers (CPDs) and 6-4 photoproducts). This system is inefficient in initiating and completing repair, such that an exposure equivalent to a 1 sunburn dose (minimal erythemal dose, MED) may not be fully repaired within 2-3 days.

Previous technologies have been designed in attempts to enhance repair of UV-induced DNA damage, but those technologies utilized pyrimidine dimer DNA glycosylases to initiate the base excision repair (BER) pathway. Pyrimidine dimer DNA glycosylases only recognize one of the two major dipyrimidine photoproducts, the CPD.

Thus, it would be advantageous to enhance the repair of DNA damage in order to rapidly remove potentially deleterious mutations.

### SUMMARY OF THE DISCLOSURE

The current disclosure provides recombinant polypeptides capable of: repairing UV-induced DNA damage in skin of a subject exposed to solar and/or UV irradiation; reducing the total tumor burden of a subject exposed to UV irradiation; and decreasing the severity of a UV-induced inflammatory response of a subject exposed to UV irradiation. A recombinant polypeptide of the disclosure forms a truncated UV damage endonuclease (hereinafter referred to as UVDE) enzyme that includes at least one heterologous targeting sequence, for instance at the carboxy-terminus (C-terminus) of the UVDE. The truncation is amino-terminal (N-terminal) to a conserved region of the UVDE required for enzymatic activity. In particular embodiments, the at least one heterologous targeting sequence includes a cell penetrating peptide (such as TAT; SEQ ID NO: 2). In particular embodiments, the at least one heterologous targeting sequence includes a nuclear localization signal (NLS), for instance a NLS (such as SEQ ID NO: 6 or 10). Optionally, the NLS is based on consensus sequences of nuclear targeting sequences of other human proteins and enzymes. In particular embodiments, the at least one heterologous targeting sequence includes both a NLS and a cell penetrating peptide. Optionally, the recombinant polypeptide further includes at least one heterologous purification (affinity) tag, such as a C-terminally linked six histidine amino acid (His6) tag. Optionally, the recombinant polypeptide further includes at least one sequence or domain (such as a SUMO domain) that is specifically recognized by a protease, for instance to enable removal of non-UVDE sequence(s) from the recombinant polypeptide.

The provided recombinant polypeptides can be encapsulated in liposomes for delivery to a subject. The current disclosure also provides recombinant polynucleotides that encode the recombinant polypeptides, vectors including the recombinant polynucleotides, and host cells including the vectors or recombinant polypeptides. The recombinant polypeptides of the disclosure may be used to: repair UV-induced DNA damage in skin of a subject; reduce the number of tumors, size of tumors and/or total tumor burden in a subject exposed to UV irradiation; treat or reduce the occurrence of a skin disorder in a subject; treat or reduce UV-induced immunosuppression in a subject in need thereof; and decrease the severity of a UV-induced inflammatory response in a subject in need thereof.

### BRIEF DESCRIPTION OF THE FIGURES

FIG. 1. Purity of UVDE-TAT-His6, UVDE-NLS-TAT-His6, and cv-pdg-NLS-His6. The purity of the DNA repair enzymes was assessed by Coomassie staining of proteins following electrophoretic separation through a denaturing 15% polyacrylamide gel. A total of 5 μg of UVDE-TAT-His6, UVDE-NLS-TAT-His6, and cv-pdg-NLS-His6 were run in lanes as indicated. Pre-stained markers were run in the left lane, with molecular weights given.

FIGS. 2A-2B. Proportion of tumor-free mice. SKH1 hairless mice were treated with control empty liposomes or liposomes containing either UVDE-TAT-His6, UVDE-NLS-TAT-His6, or cv-pdg-NLS-His6 1 hr prior to irradiation. Mice were irradiated in individual chambers covered with quartz glass that allowed full UVB transmission of the increasing UVB doses delivered on a M, W, F schedule. FIG. 2A and FIG. 2B show the proportion of mice in each group that have not developed a 2 mm tumor at specified time points across the study period. FIG. 2A: Control empty liposome formulation (solid line), liposomes containing UVDE-TAT-His6 (dashed line), and liposomes containing UVDE-NLS-TAT-His6 (dotted line). FIG. 2B: Control empty liposome formulation (solid line) and liposomes containing cv-pdg-NLS (dashed line). Proportions of tumor-free mice in each group were plotted with Kaplan-Meier curves and compared using the log-rank test. Mice that never developed a 2 mm tumor during the study period were represented as plus (+) signs on the plots. Severe ulceration meeting the criteria for mandatory euthanasia never occurred for tumors ≤2 mm.

FIGS. 3A-3C. Suppression of tumor formation in SKH1 hairless mice treated with liposomes containing UVDE-TAT-His6 and UVDE-NLS-TAT-His6. Three representative photographs of mice from each treatment group are shown: control empty liposome formulation (FIG. 3A), liposomes containing UVDE-TAT-His6 (FIG. 3B), and liposomes containing UVDE-NLS-TAT-His6 (FIG. 3C) at 23 weeks (cumulative dose of 311 kJ/m²).

FIGS. 4A-4D. Analyses of total tumor size at 23 and 33 weeks of UVB irradiation. To assess the extent of protection afforded by topical delivery of UVDE-TAT-His6 and UVDE-NLS-TAT-His6 (FIGS. 4A and 4C) and cv-pdg-NLS-His6 (FIGS. 4B and 4D), respectively, analyses of the aggregate size of UVB-induced tumors for each mouse are shown for data collected at 23 (FIGS. 4A and 4B) and 33 (FIGS. 4C and 4D) weeks of irradiation. All tumors were measured and the sum of all tumor diameters was calculated for each mouse to represent total tumor burden. Final measured (pre-death) tumor sizes were used for mice that required euthanasia prior to weeks 23 or 33. At each time point of interest, the sample distribution of total tumor size for each of the 4 groups of mice is displayed as a box plot with a horizontal line inside the box depicting the median, a diamond symbol denoting the mean, and outliers represented as circles outside the box. Group comparisons of total tumor burden at each of the two time points were conducted using analysis of variance (ANOVA).

FIG. 5. Analyses of total tumor size by treatment group (including imputed values) analyzed at 18 weeks, 21 weeks, 24 weeks, 28 weeks, 30 weeks and 33 weeks of UVB irradiation. To assess the extent of protection afforded by topical delivery of UVDE-TAT-His6, UVDE-NLS-TAT-His6 and cv-pdg-NLS-His6, analyses of the aggregate size of UVB-induced tumors for each mouse are shown for data collected at 18, 21, 24, 28, 30 and 33 weeks of irradiation. All tumors were measured and the sum of all tumor diameters was calculated for each mouse to represent total tumor burden. Final measured (pre-death) tumor sizes were used for mice that required euthanasia prior to weeks 18, 21, 24, 28, 30 and 33. At each time point of interest, the sample distribution of total tumor size for each of the 4 groups is displayed as a box plot with a horizontal line inside the box depicting the median and outliers represented as circles outside the box.

FIGS. 6A-6B. Kaplan-Meier plot of survival. Throughout the course of UVB irradiation, mice were routinely monitored for tumors that became ulcerated or were >8 mm in diameter. Mice that met either of these criteria were euthanized, which served as the death endpoint for survival analysis. The proportion of mice not developing a tumor that met the above criteria is plotted across the study period. FIG. 6A: control empty liposome formulation (solid line), liposomes containing UVDE-TAT-His6 (dashed line), and liposomes containing UVDE-NLS-TAT-His6 (dotted line). FIG. 6B: control empty liposome formulation (solid line) and liposomes containing cv-pdg-NLS-His6 (dashed line). The plotted survival proportions were estimated using the Kaplan-Meier method and statistically compared with the log-rank test. Mice that did not meet the criteria for euthanasia by the end of the 33-week study period (i.e., censored observations) were represented with plus (+) signs. Since all mice were analyzed until either (i) euthanasia due to tumor criteria or (ii) the study ended, there was no censoring prior to the last observed time point at 33 weeks, and Kaplan-Meier estimates were equal to the sample proportions of mice remaining alive at any given time.

FIGS. 7A-7C. Representative histology of tumors formed following UVB irradiation. At the time of euthanasia, representative skin tissue samples were harvested, prepared for histologic analyses, and photographed at 5 and 20× in left and right panels, respectively. Representative tumors are shown from skin tissues containing tumors from the following groups: control empty liposomes (FIG. 7A), liposomes containing UVDE-TAT-His6 (FIG. 7B), and liposomes containing UVDE-NLS-TAT-His6 (FIG. 7C). No qualitative differences were noted in the general characteristics of the tumors that were formed as a result of the cumulative UVB irradiations. All inset bars represent 50 μM.

FIGS. 8A-8C. Schematics illustrating a full-length Schizosaccharomyces pombe UVDE protein (FIG. 8A), a truncated tagged UVDE protein of the present disclosure (FIG. 8B), and a UVDE expression construct containing a protease-specific region that permits removal of domain(s) not needed in the final therapeutic preparation (FIG. 8C). FIG. 8A: The uve1 gene encodes a 599 amino acid protein containing a putative nuclear localization signal (NLS) region (amino acids 99-116), a coiled coil region (amino acids 155-185), and a conserved region (amino acids 250-527) similar to regions found in the N. crassa and B. subtilis UVDE functional homologs that is thought to be required for enzymatic activity (Siede, W. & Doetsch, P. W. (Eds.). DNA damage recognition. CRC Press, 2005). FIG. 8B: The present disclosure uses an N-terminal truncated version of S. pombe UVDE lacking the first 228 amino acids (the truncation is abbreviated ‘UVDE’ herein) with a cell penetrating peptide (TAT)-hexahistidine tag (TAT-6xHis) or NLS-TAT-hexahistidine tag (NLS-TAT-6xHis) linked to the C-terminus of the truncated UVDE. For the illustrated TAT-6xHis (positions 371-389 of SEQ ID NO: 12) and NLS-TAT-6xHis (positions 371-397 of SEQ ID NO: 14) sequences, the last amino acid of UVDE (K) is bolded, Leucine residues from a linker are underlined, the TAT sequence is double-underlined, and the hexahistidine tag is italicized. FIG. 8C illustrates the structure of an expression construct in which the recombinant enzyme includes a start codon (Met), followed by a sequence coding for amino acids dictating binding and cleavage site for a protease (in the illustrated instance, SUMO), followed by the UVDE coding sequence, followed by a TAT sequence (or other cell penetrating domain), and ending in a stop codon (illustrated as TAA). Following isolation of this type of chimeric polypeptide, the cognate protease (e.g., ULP-1 if SUMO is used) is used to cleave the polypeptide at the junction with the protease recognition sequence and the UVDE-TAT purified with no His6 sequences remaining.

### SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “Sequence Listing.txt” created on or about Apr. 16, 2020, with a file size of 52 KB, contains the sequence listing for this application and is hereby incorporated by reference in its entirety. In the accompanying Sequence Listing:

| SEQ ID | |
|---|---|
| NO: | DESCRIPTION OF SEQUENCE |
| 1 | Nucleotide sequence encoding a protein transduction |
| | domain TAT (transactivator of transcription) from |
| | human immunodeficiency virus set forth in |
| | SEQ ID NO: 2 |
| 2 | Amino acid sequence for a TAT peptide |
| 3 | Nucleotide sequence encoding a nuclear localization |
| | signal (NLS) and TAT set |
| | forth in SEQ ID NO: 4 |
| 4 | Amino acid sequence for an NLS-TAT |
| 5 | Nucleotide sequence encoding a nuclear localization |
| | signal (NLS) set forth in |
| | SEQ ID NO: 6 used in the UVDE-NLS-TAT-His6 expression |
| | construct |
| 6 | Amino acid sequence of NLS used in the UVDE-NLS- |
| | TAT-His6 expression construct |
| 7 | Amino acid sequence of a linker between the last |
| | codon of an S. pombe UVDE |
| | and a hexahistidine tag |
| 8 | Amino acid sequence for a hexahistidine tag |
| 9 | Nucleotide sequence encoding a nuclear localization |
| | signal (NLS) set forth in |
| | SEQ ID NO: 10 used in the cv-pdg-NLS-His6 expression |
| | construct |
| 10 | Amino acid sequence for an NLS used in the cv-pdg- |
| | NLS-His6 expression construct |
| 11 | Nucleotide sequence encoding UVDE-TAT-His6 set forth |
| | in SEQ ID NO: 12; |
| | positions 1111-1116 a restriction site |
| | positions 1-1113 coding sequence for the truncated |
| | UVDE described herein |
| | positions 1114-1116 coding sequence for a linker |
| | residue |
| | positions 1117-1149 coding sequence fora TAT peptide |
| | of SEQ ID NO: 2 |
| | positions 1150-1167 coding sequence for a hexahistidine |
| | tag. |
| 12 | Amino acid sequence for a UVDE-TAT-His6 recombinant |
| | polypeptide |
| | positions 1-371 truncated UVDE described herein |
| | positions 372 Leucine denotes a linker residue |
| | positions 373-383 sequence for a TAT peptide of |
| | SEQ ID NO: 2 |
| | positions 384-389 the sequence for a hexahistidine |
| | tag |
| 13 | Nucleotide sequence encoding UVDE-NLS-TAT-His6 set |
| | forth in SEQ ID NO: 14 |
| | positions 1111-1116 a restriction site |
| | positions 1-1113 coding sequence for the truncated |
| | UVDE described herein |
| | positions 1114-1116 coding sequences for linker |
| | residues |
| | positions 1117-1137 coding sequence for an NLS of |
| | SEQ ID NO: 6 |
| | positions 1138-1173 coding sequence for a TAT |
| | peptide of SEQ ID NO: 2 |
| | positions 1174-1191 coding sequence for a |
| | hexahistidine tag |
| 14 | Amino acid sequence for a UVDE-NLS-TAT-His6 |
| | recombinant polypeptide |
| | positions 1-371 sequence for the truncated |
| | UVDE described herein |
| | positions 372 and 380 leucine linker residues |
| | positions 373-379 the sequence for an NLS of |
| | SEQ ID NO: 6, |
| | positions 381-391 the sequence for a TAT |
| | peptide of SEQ ID NO: 2 |
| | positions 392-397 the sequence for a |
| | hexahistidine tag |
| 15 | Nucleotide sequence encoding a full-length |
| | Schizosaccharomyces pombe |
| | UVDE set forth in SEQ ID NO: 16 (NCBI Ref |
| | Seq NM_001022085.2) |
| 16 | Amino acid sequence for a full-length |
| | Schizosaccharomyces pombe UVDE |
| | (NCBI Ref Seq: NP_596165.1) |
| 17 | Amino acid sequence for an SV40 large |
| | T antigen nuclear localization signal |
| | (NLS) |
| 18 | Amino acid sequence for a c-Myc nuclear |
| | localization signal (NLS) |
| 19 | Amino acid sequence for a putative consensus |
| | sequence of the bipartite nuclear |
| | localization signal (NLS) |
| | (K/R)(K/R)X₁₀₋₁₂(K/R)_{3/5} |
| | (K/R)_{3/5} represents at least three of either |
| | lysine or arginine of five consecutive |
| | amino acids |
| 20-61 | Amino acid sequences of exemplary nuclear |
| | localization signal (NLS) sequences |
| | (see Table 1) |
| 62 | Nucleotide sequence for a hexahistidine tag |
| 63 | Nucleotide sequence encoding a truncated |
| | Schizosaccharomyces pombe UVDE |
| | lacking amino acid residues 1-228 as set |
| | forth in SEQ ID NO: 64 |
| 64 | Amino acid sequence for a truncated |
| | Schizosaccharomyces pombe UVDE |
| | lacking amino acid residues 1-228 |
| 65 | Amino acid sequence of a linker between the |
| | last codon of an S. pombe UVDE |
| | and a hexahistidine tag removed during |
| | cloning of S. pombe UVDE-TAT-6xHis |
| 66-83 | Amino acid sequences of exemplary cell- |
| | penetrating peptides (see Table 2) |
| 84 | Acid sequence of His6SUMO-UVDE-TAT |
| | positions 2-7 sequence for a hexahistidine |
| | tag |
| | positions 8-107 sequence for a SUMO domain |
| | positions 108-478 sequence for a truncated |
| | UVDE as described |
| | positions 479-490 sequence for a TAT cell |
| | penetrating peptide |
| 85 | Nucleotide sequence of His6SUMO-UVDE-TAT |
| | positions 4-21 coding sequence for a |
| | hexahistidine tag |
| | positions 22-321 coding sequence for a |
| | SUMO domain |
| | positions 322-1434 coding sequence for a |
| | truncated UVDE as described |
| | positions 1435-1470 coding sequence for a |
| | TAT cell penetrating peptide |

### DETAILED DESCRIPTION

Non-melanoma skin cancers (NMSCs), including basal cell carcinoma (BCC) and squamous cell carcinoma (SCC) are the most prevalent types of human cancers, affecting over five million people in the United States annually, and costing billions of dollars for health care and loss of work (Guy et al., 2012, supra; Wu et al., 2015, supra; Bickers et al., 2006, supra). In addition to high rates of disease in the general population, organ transplant patients have a greater than 50-fold increase in the incidence of NMSC, with increased risk of metastasis (Reichrath, Adv Exp Med Biol 810, 253-271, 2014; Ruiz & Hsieh, J Clin Aesthet Dermatol 8, 16-19, 2015; Abgrall et al., Anticancer Res 22, 3597-3604, 2002). Current methods for treatment of NMSC, including surgical resection of the tumor, are associated with considerable pain and morbidity. Given these exceptionally high incidence rates, strategies to prevent skin cancer have predominantly focused on recommendations for sun avoidance, restricted access of youth to tanning beds, the use of broad spectrum UVA and UVB sunscreens, and application of topical anti-oxidants. However, these recommendations have not sufficiently diminished the prevalence of NMSC, and development of novel methods to reduce or prevent NMSCs would not only alleviate suffering, but also substantially reduce health care costs.

Exposure to UV irradiation in sunlight causes NMSC by inducing DNA damage that if replicated, leads to mutations in genes such as KRAS or TP53 (Sarasin, Mutat Res 428, 5-10, 1999). Two common types of UVB-induced DNA damage are cyclobutane pyrimidine dimers (CPDs) and 6-4 photoproducts (6-4 PPs). 6-4 PPs are more rapidly repaired than CPDs in both fibroblasts and keratinocytes (Courdavault et al., DNA Repair (Amst) 4:836-844, 2005). In response to broad-spectrum UV-induced DNA damage, skin cells transiently arrest progression through the cell cycle to allow DNA repair, or in the case of irreparable DNA damage, die via apoptosis. CPDs account for at least 80% of UVB-induced mutagenesis but contribute only modestly to cytotoxicity in a mammalian cell model; however, 6-4 PPs are minimally mutagenic but highly cytotoxic (Lo et al., BMC Cancer 5:135, 2005, doi:10.1186/1471-2407-5-135; You et al., J Biol Chem 276:44688-44694, 2001). Thus, it can be concluded that coordination of these responses is crucial for protection against skin carcinogenesis.

Humans possess only one mechanism for repairing dipyrimidine DNA lesions, the nucleotide excision repair (NER) pathway. The importance of this DNA repair system in limiting NMSC is best demonstrated by the clinical sequela of patients suffering from the autosomal recessive genetic disorder Xeroderma Pigmentosum (XP). These patients have defects in either NER or DNA translesion synthesis (TLS) of CPDs, either of which will significantly increase the risk for development of NMSC. In fact, XP patients have greater than a 1000-fold increased risk of developing skin cancer before the age of 20 compared to the general population (Kraemer et al., J Invest Dermatol 103, 96S-101S, 1994).

In contrast to humans, some organisms can utilize the base excision repair (BER) pathway in addition to NER, for repairing UV-induced DNA lesions. Humans have all of the enzymes necessary for completing BER, but lack an enzyme, a pyrimidine dimer-specific DNA glycosylase (pdg) to recognize CPDs and initiate the cascade. Therefore, one strategy for enhancing repair of CPDs in human skin cells has been to deliver, or express, the bacteriophage T4 pdg in these cells to provide an enzyme that repairs CPDs (Lloyd, Mutat Res 577:77-91, 2005). Collectively, several studies have shown that T4-pdg could not only initiate repair following UV damage of XP cells (Tanaka et al., Proc Natl Acad Sci U S A 72:4071-4075, 1975), but also increase survival in XP cells (Francis et al., Photochem Photobiol 72:365-373, 2000). Further, T4-pdg protein has been encapsulated into a liposomal delivery vehicle for use in studies on murine and human skin (Yarosh et al., J Invest Dermatol 103:461-468, 1994; Yarosh et al., Photodermatol Photoimmunol Photomed 12:122-130, 1996). Delivery of T4-pdg in mouse models increased the rate of CPD removal, reduced the frequency of SCC, and minimized UVB-induced immune suppression. Results of clinical trials with XP patients using topically delivered T4-pdg showed that new pre-cancerous lesions (actinic keratosis) were reduced by 68% and new cases of BCC were reduced by 30% compared to patients treated with placebo lotion (Yarosh et al., Lancet 357, 926-929, 2001).

Although these data were encouraging, a potential limitation for the use of any pdg is that their substrate specificities do not include recognition of 6-4 PPs. However, the UV endonuclease (UVDE) from Schizosaccharomyces pombe has a very broad substrate specificity which includes both CPDs and 6-4 PPs and is known to initiate the nucleotide incision repair (NIR) pathway (Avery et al., Nucleic Acids Res 27, 2256-2264, 1999; Kaur & Doetsch, Biochemistry 39, 5788-5796, 2000). To increase its solubility while retaining full catalytic activity, previous studies have characterized a soluble form of UVDE in which the N-terminal 228 amino acids were deleted (Δ228-UVDE). Since the truncated form of UVDE was exclusively used in all studies described herein, it is abbreviated UVDE, but refers to Δ228-UVDE. Although this enzyme has not been previously used for in vivo repair and carcinogenesis studies, the increased substrate specificity makes it an ideal candidate for use in investigations of UV-induced carcinogenesis. Further limitations of the original study design with T4-pdg were that it poorly localized to the nucleus and once delivered to the skin, could not redistribute to cells in the immediate vicinity. To address these challenges, an experimental strategy included adding a cell-penetrating peptide from HIV Tat transcriptional activator (TAT) (Joliot & Prochiantz, Nat Cell Biol 6:189-196, 2004) that facilitates migration of the associated protein between cells and to engineer a nuclear localization signal (NLS) onto the repair enzyme. Additionally, the cv-pdg enzyme was engineered to be expressed with a C-terminal NLS. Reported herein are studies which characterize topical delivery of TAT-His6-, NLS-TAT-His6-modified UVDE, and cv-pdg-NLS-His6, including whether these differentially modulate UVB-induced carcinogenesis in a SKH1 hairless mouse model.

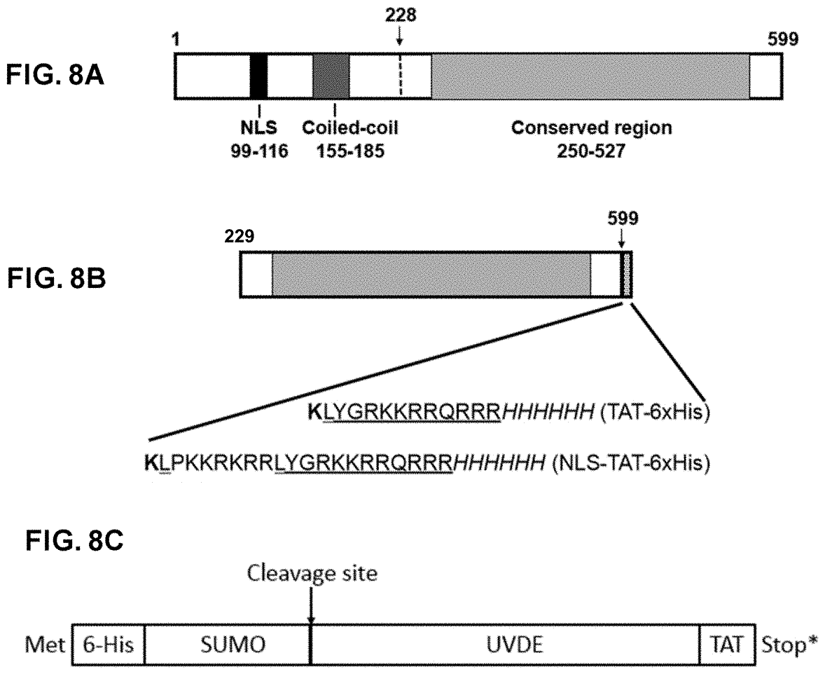

To enhance the DNA repair capacity of human cells following UV exposure, specific embodiments of the technology described herein utilize a multifunctional DNA repair enzyme from the yeast Schizosaccharomyces pombe, UV Damage Endonuclease (UVDE), that is able to recognize both cyclobutane pyrimidine dimers (CPDs) and 6-4 photoproducts. In order to enhance the localization of UVDE to the nucleus, the following have been done in various embodiments: 1) the UVDE protein lacking the first 228 amino acids of SEQ ID NO: 16 has been engineered to have a nuclear localization signal (NLS) and/or cell penetrating peptide TAT or TAT, for instance at the C-terminus of the enzyme (see FIG. 8B); 2) genes encoding UVDE-TAT-His6 and UVDE-NLS-TAT-His6 have been cloned into bacterial expression vectors from which the expression of the tagged UVDE enzymes can be controlled; 3) large scale manufacturing and purification of the UVDE-TAT-His6 and UVDE-NLS-TAT-His6 enzymes have been developed; 4) conditions have been established to encapsulate the purified UVDE-TAT-His6 and UVDE-NLS-TAT-His6 into liposomes to be delivered as active enzymes into skin; 5) it has been demonstrated that topical application of UVDE-TAT-His6 or UVDE-NLS-TAT-His6 in a minipig model can repair the DNA damage that is produced by a 2-MED (minimal erythemal) dose) dose within 6 hr post exposure; 6) it has been demonstrated that topical application of UVDE-TAT-His6 or UVDE-NLS-TAT-His6 can reduce the amount of inflammatory circulating white blood cells (lymphocytes, monocytes, and eosinophils), a result indicative of preventing UV-induced immunosuppression; 7) it has been demonstrated that topical delivery of UVDE-TAT-His6 and UVDE-NLS-TAT-His6 to the skin of hairless mice can significantly reduce the severity of skin cancer caused by UVB light exposure.

Also described are recombinant polypeptides (and nucleic acids for expressing such polypeptides) that include at least one sequence or domain (such as a SUMO domain) that is specifically recognized by a protease, for instance to enable removal of non-UVDE sequence(s) from the recombinant polypeptide. SEQ ID NOs: 84 and 85, as well as FIG. 8C, describe an example of such embodiments.

In head-to-head DNA repair efficacy trials using the mini-pig model, the UVDE-TAT-His6 or UVDE-NLS-TAT-His6 enzyme substantially reduces the amount of circulating lymphocytes, monocytes, and/or eosinophils compared to cv-pdg-NLS-His6. Substantially reducing the amount of circulating lymphocytes, monocytes, and/or eosinophils means at least a 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more reduction in the amount of circulating lymphocytes, monocytes, and/or eosinophils when comparing the effects of UVDE-TAT-His6 or UVDE-NLS-TAT-His6 versus cv-pdg as assayed in the Gottingen mini-pig skin model. Thus, the UVDE-NLS-His6 enzyme has superior properties relative to those of the pyrimidine dimer glycosylase.

There are numerous major differences between enzymes used in previous therapeutic strategies and those described herein. First, pyrimidine dimer glycosylases (pdg) and UV endonucleases have no significant similarity at the nucleotide and amino acid sequence level; they are completely different enzymes. Further, the organisms that produce pyrimidine dimer glycosylases are different (and non-overlapping) with organisms that produce UV endonucleases described herein. Pdgs (that recognize cyclobutane pyrimidine dimers and catalyze cleavage of the glycosyl bond attaching the 5′ pyrimidine of the dimer to its corresponding deoxyribose and further catalyze a β-elimination reaction at the 5′ deoxyribose resulting in a single-stranded break in the phosphodiester backbone) are found in bacteriophage T4 and Chlorella, for instance (Bailly et al., Biochem J. 259(3):751-759, 1989; Dodson et al., Biochemistry. 32(32):8284-90, 1993; Schrock & Lloyd, J Biol Chem. 268(2):880-6, 1993; Schrock & Lloyd, J Biol Chem. 266(26):17631-9, 1991; McCullough et al., J Biol Chem. 273(21):13136-42, 1998). In contrast, UV endonucleases are found in Schizosaccharomyces pombe and Neurospora crassa; other putative UV endonucleases have been identified by DNA sequence alignment in Bacillus megaterium, Thermus thermophilus, and Halobacterium marismortui.

UV endonuclease enzymes are further distinguishable from pdgs in that they recognize a broader subset of DNA damages and they generate direct DNA strand breaks immediately 5′ to the pyrimidine dimer or 6-4 photoproduct (or other DNA damage), producing a 3′ hydroxyl and a 5′ phosphate (Kaur & Doetsch, Biochemistry. 39(19):5788-96, 2000; Avery et al., Nucleic Acids Res. 27(11):2256-64, 1999; Kaur et al., Biochemistry. 37(33):11599-604, 1998). The substrate specificity of pdgs is limited to cyclobutane pyrimidine dimers and ring-fragmented purines, including formamidopyrimidine (Fapy)-dA and Fapy-dG (McMillan et al., J Virol. 40(1):211-23, 1981; Friedberg et al., J Virol. 13(5):953-9, 1974; Dizdaroglu et al., Mutat Res. 362(1):1-8, 1996; Jaruga et al., Photochem Photobiol. 75(2):85-91, 2002). UVDE has a much broader substrate specificity including cyclobutane pyrimidine dimers, 6-4 photoproducts, cis-platin-induced dG-dG intrastrand DNA crosslinks, 12 different mismatched DNA nucleotide combinations, DNAs that have been treated with intercalating agents such as acridine dyes, and potentially DNAs that have been modified with alkylating agents that produce significant distortions in the DNA duplex structure (Kaur & Doetsch, Biochemistry. 39(19):5788-96, 2000; Avery et al., Nucleic Acids Res. 27(11):2256-64, 1999; Kaur et al., Biochemistry. 37(33):11599-604, 1998).

The active site residues of the pyrimidine dimer glycosylases are different than the UV endonuclease enzymes described herein. The active site residues for the pdgs are always the N-terminal α-amino group and an acidic acid residue (Glu) at position 23 in which these two amino acids are in close proximity to constitute the essential elements for catalyzing the dual chemical reactions (Bailly et al., Biochem J. 259(3):751-759, 1989; Dodson et al., Biochemistry. 32(32):8284-90, 1993; Schrock & Lloyd, J Biol Chem. 268(2):880-6, 1993; Schrock & Lloyd, J Biol Chem. 266(26):17631-9, 1991; McCullough et al., J Biol Chem. 273(21):13136-42, 1998; Morikawa et al., Science. 256(5056):523-6, 1992). No metal ion(s) are required for the activity of the pdgs, such that they are fully functional in the presence of metal chelating agents. The active site residues for UVDE have been inferred from the crystal structure of a biochemically active fragment of UVDE from T. thermophilus and is inferred to be conserved among UVDEs from organisms such as S. pombe (Paspaleva et al., Structure. 15(10):1316-24, 2007). The mechanism of DNA damage incision by the UVDEs is metal (e.g., magnesium, manganese) catalyzed, presumably requiring three metals that are bound by residues in the C-terminal portion of the enzyme. It is expected that the requirements are a general base to position and activate the nucleophile to achieve an in-line attack of the phosphate, a general acid to protonate the leaving group, and a Lewis acid to stabilize the pentacovalent phosphoanion transition state. In UVDE, this is predicted to be supplied by metal coordination from the following: i) an octahedrally coordinated metal using four coordinating residues, H231, D200, E269, and E175; ii) a distorted bipyramidal coordination of the second metal ion by His-101, His-143, and Glu-175, and iii) the third metal ion coordinated by one oxygen atom from the phosphate, His-244, His203, and one water molecule (Paspaleva et al., Structure. 15(10):1316-24, 2007).

As a result of structural differences, the chemical mechanism of action by which the pyrimidine dimer glycosylases initiate repair at sites of DNA damage is totally different from the UV endonuclease enzymes described herein. To restate, pdgs work via an activated N-terminal α-amino group (constituting the nucleophile) attacking the C1′ of the 5′ deoxyribose of the pyrimidine dimer (Dodson et al., Biochemistry. 32(32):8284-90, 1993; Schrock & Lloyd, J Biol Chem. 266(26):17631-9, 1991). UVDEs catalyze a 3-metal coordinated in-line attack of the phosphate 5′ to the damaged DNA substrate (Paspaleva et al., Structure. 15(10):1316-24, 2007). Similarly, the three-dimensional structures of the pyrimidine dimer glycosylases are different than the enzymes described herein.

The structures of the incised DNAs following catalysis by the pyrimidine dimer glycosylases are different than those produced by the UV endonuclease enzymes described herein. The incised DNA created by pdgs is a 3′ α, β-unsaturated aldehyde and a 5′ phosphate; in a subset of reactions, pdgs can also catalyze a further δ-elimination reaction that removes the 3′ α, β-unsaturated aldehyde and leaves a 3′ phosphate, as well as the 5′ phosphate. UVDEs produce a 3′ hydroxyl and a 5′ phosphate. The subsequent DNA repair pathways that complete the repair process for the pyrimidine dimer glycosylases are also different from the enzymes described herein. Pdgs work through the base excision repair pathway, while UVDEs work through an alternative excision repair pathway.

The potential clinical usefulness of pyrimidine dimer glycosylases is more limited because these enzymes do not recognize one of the major DNA photoproducts of solar and UV irradiation (the 6-4 photoproduct). This is critically important since the predominant location of 6-4 photoproducts is in sites of active DNA transcription in cells. Since these photoproducts block transcription, it is of critical importance to preferentially repair these sites. The previous technologies (that employ a pyrimidine dimer glycosylase) do not initiate repair of 6-4 photoproducts, but the UV endonucleases described herein will and thus are preferable. It would be advantageous to enhance the repair of both major forms of solar and UV-light induced DNA damage in order to rapidly remove potentially deleterious mutations.

The present disclosure provides polypeptides including targeting sequences that have the ability to remove cyclobutane pyrimidine dimers (CPDs) and/or (6-4) photoproducts (6-4 PPs) from DNA. The term “polypeptide” refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, and phosphorylations.

The term “ultraviolet damage endonuclease”, “UV damage endonuclease” or “UVDE” refers to a polypeptide that has the ability to remove CPDs and/or 6-4 PPs from DNA. A polypeptide that has the ability to remove CPDs and/or 6-4 PPs from DNA has “UV damage endonuclease activity” or “UVDE activity.” In particular embodiments, “UVDE” refers to an amino-terminal (N-terminal) truncated UVDE, where the truncation is amino-terminal (N-terminal) to a conserved region of the UVDE required for enzymatic activity (for example, Δ228-UVDE is a Schizosaccharomyces pombe UVDE lacking the first 228 amino acid residues of a full-length Schizosaccharomyces pombe UVDE encoded by SEQ ID NO: 16; Δ228-UVDE is encoded by nucleotide sequence SEQ ID NO: 63 shown in FIGS. 15 and Δ228-UVDE amino acid sequence is SEQ ID NO: 64 shown in FIG. 16). In particular embodiments, “UVDE” also includes full-length homologs of UVDE enzymes from different organisms, such as bacteria, fungi and mammals. In particular embodiments, “UVDE” also includes N-terminal truncated homologs of UVDE enzymes from different organisms, such as bacteria, fungi and mammals, that exhibit similar biological activity to the Schizosaccharomyces pombe UVDE lacking the first 228 amino acid residues (Δ228-UVDE) of a full-length Schizosaccharomyces pombe UVDE encoded by SEQ ID NO: 16. Whether a polypeptide has UVDE activity or has similar biological activity to S. pombe Δ228-UVDE can be determined by, for example, measuring the ability of the polypeptide to cleave dipyrimidine (a.k.a., bipyrimidine) photoproduct substrates. Such methods are known in the art (see, e.g., U.S. Pat. No. 6,368,594). In particular embodiments, similar biological activity between S. pombe Δ228-UVDE and a full-length or N-terminal truncated UVDE homolog means that there is no statistically significant difference in number of tumors, size of tumors and/or total tumor burden; frequency of or time to onset of skin cancers; and/or risk of death (hazard ratios) in UV irradiated SKH1 hairless mice treated with each UVDE in a UVB-induced carcinogenesis study as described below. In particular embodiments, similar biological activity between S. pombe Δ228-UVDE and a full-length or N-terminal truncated UVDE homolog means that there is no statistically significant difference in the amount of circulating lymphocytes, monocytes and eosinophils in UV irradiated minipigs treated with each UVDE in a Gottingen minipig study as described below. The UVDE polypeptides of the present disclosure can recognize a wide variety of DNA damage and distortions, such as pyrimidine dimers; non-UV photoproduct dimer lesions, e.g., platinum-DNA lesions; abasic sites; uracil and dihydrouracil (DHU) lesions; and base mismatches.

Homologs of UVDE are present in many fungal species but also in a number of bacteria, such as Bacillus subtilis and the thermophilic bacterium Thermus thermophilus (e.g., GenBank Accession No. WP_011174507.1; RCSB Protein Data Bank ID 2j6v). Examples of polypeptides having UVDE activity include amino acid sequences present in Schizosaccharomyces pombe (Uve1p; GenBank Accession No. NP_596165.1; SEQ ID NO: 16), Neurospora crassa (GenBank Accession No. BAA 74539), and B. subtilis (GenBank Accession No. 249782).

The crystal structure of a UVDE from Thermus thermophilus has been determined (Paspaleva et al., Structure, 15(10): 1316-1324, 2007). The general structure of the UVDE protein includes a single-domain TIM barrel (lacking the α8 helix) of the prototypical TIM-barrel fold. The TIM barrel is a conserved protein fold consisting of 8 α-helices and 8 parallel β-strands and is considered one of the most common protein folds. A distinct crescent-shaped groove formed by the C-terminal end of the TIM barrel forms the enzyme active site. The UVDE is classified as a member of the TIM-barrel family of divalent metal-dependent enzymes due to the three anomalously scattering metal ions, located closely to the C terminus and due to the close proximity of the protein's N and C termini. The UVDE structure reveals a novel use of the TIM barrel fold for binding DNA for damage recognition and catalysis. The enzyme must bind and scan normal DNA via electrostatic complementarity and hydrogen bonding to the DNA phosphate backbone from β barrel loops and α-helical dipoles identically positioned by the α8β8 framework. DNA damage detection proceeds by insertion of side chains from minor groove recognition loops to provide DNA backbone compression and flipping of the target apurinic/apyrimidinic (AP) site and its opposing nucleotide out of the helix.

The uve1 gene of Schizosaccharomyces pombe encodes a 599 amino acid full-length UVDE protein (SEQ ID NO: 16) containing a putative nuclear localization signal (NLS) region (amino acids 99-116), a coiled coil region (amino acids 155-185), and a conserved region (amino acids 250-527) similar to regions found in the N. crassa and B. subtilis UVDE functional homologs that is thought to be required for enzymatic activity (FIG. 8A).

The UVDE polypeptides of the present disclosure also include at least one heterologous targeting sequence. The term “targeting sequence” is a polypeptide that is linked to a polypeptide having UVDE activity. The targeting sequence can be heterologous, which refers to a targeting sequence that is not normally linked to the polypeptide having UVDE activity. The heterologous targeting sequences can be linked to a polypeptide having UVDE activity at the amino-terminal or carboxy-terminal end of the UVDE polypeptide. Targeting sequences can, for example, cause the polypeptide to which they are linked to migrate from the cytoplasm of a cell to an organelle or cause the polypeptide to which they are linked to reach the cytoplasmic and/or nuclear compartments in live cells after internalization. Methods to confirm that the polypeptide has been correctly targeted to an organelle or to a cell are known in the art and are described elsewhere herein.

In particular embodiments, the targeting sequence is a nuclear localization signal (NLS) sequence. NLSs are amino acid sequences that target polypeptides into the nucleus. Targeting to the nucleus is enabled by binding of the NLSs to their receptors, known as importins (karyopherins). Nuclear import of proteins is generally initiated by the formation of a ternary complex with importin α, importin β1, and a cargo (such as a polypeptide), where importin β1 docks the complex to the nuclear pore complex to release the cargo into the nucleus through the binding of Ran-GTP to importin β1. In the importin α/β pathway, importin α serves as an adaptor that links cargos and importin β1 and recognizes NLSs within the cargos. Importin a recognizes two classes of NLSs, known as classical NLSs: monopartite NLSs having a single cluster of basic amino acid residues and bipartite NLSs having two clusters of basic amino acids separated by a 10-12-amino acid linker. Further, there are two types of monopartite NLSs; one has at least four consecutive basic amino acids, exemplified by the SV40 large T antigen NLS (PKKKRKV, SEQ ID NO: 17), whereas the other has only three basic amino acids and is represented by K(K/R)X(K/R) as a putative consensus sequence. The latter is exemplified by the c-Myc NLS (PAAKRVKLD, SEQ ID NO: 18). A putative consensus sequence of the bipartite NLS has been defined as (K/R)(K/R)X₁₀₋₁₂(K/R)_{3/5} (SEQ ID NO: 19), where (K/R)3/5 represents at least three of either lysine or arginine of five consecutive amino acids, in which the linker region has been found to be tolerant to amino acid conversion (Dingwall & Laskey, Trends Biochem. Sci. 16, 478-481, 1991; Robbins et al., Cell 64, 615-623, 1991). Although the putative consensus sequences of the classical NLSs have been defined, there are a number of experimentally defined NLSs that do not match the consensus sequences. An NLS can be present in any location in a polypeptide of the present disclosure provided the presence of the NLS does not inhibit the UVDE activity of the polypeptide after the UVDE polypeptide is delivered to the nucleus. In particular embodiments, an NLS is present at the carboxy terminal (C-terminal) end of a UVDE. In particular embodiments, the NLSs are amino acid sequences selected from SEQ ID NOs: 6, 10, 17, 18, and 19-61 (see Table 1).

TABLE 1
Exemplary NLS Sequences

| SEQ | |
|---|---|
| ID | |
| NO: | Sequence |
| 19 | (K/R)(K/R)X₁₀₋₁₂(K/R)_{3/5} |
| 20 | PKKKRMV |
| 21 | PKKKRKVEDP |
| 22 | PKKGSKKA |
| 23 | PKTKRKV |
| 24 | CGGPKKKRKVG |
| 25 | PKKKIKV |
| 26 | CYDDEATADSQHSTPPKKKRKVEDPKDFESELLS |
| 27 | CGYGPKKKRKVGG |
| 28 | CGYGVSRKRPRPG |
| 29 | APTKRKGS |
| 30 | APKRKSGVSKC |
| 31 | PNKKKRK |
| 32 | EEDGPQKKKRRL |
| 33 | GKKRSKA |
| 34 | CGGLSSKRPRP |
| 35 | LKDKDAKKSKQE |
| 36 | GNKAKRQRST |
| 37 | PFLDRLRRDQK |
| 38 | SVTKKRKLE |
| 39 | SASKRRRLE |
| 40 | PPKKRMRRRIE |
| 41 | YRKCLQAGMNLEARKTKK |
| 42 | KIKGIQQATA |
| 43 | RKDRRGGRMLKHKRQRD |
| 44 | DGEGRGEVGSAGDMRAMINACIDNLWPSPLMIKRSK |
| 45 | RKFKKFNK |
| 46 | PLLKKIKQ |
| 47 | PPQKKIKS |
| 48 | PQPKKKP |
| 49 | SKRVAKRKL |
| 50 | MTGSKTRKHRGSGA |
| 51 | RHRKHP |
| 52 | KRRKHP |
| 53 | KYRKHP |
| 54 | KHRRHP |
| 55 | KHKKHP |
| 56 | RHLKHP |
| 57 | PETTWRRRGRSPRRRTP |
| 58 | SPRRRRSPRRRRSQS |
| 59 | ASKSRKRKL |
| 60 | GGLCSARLHRHALLAT |
| 61 | DTREKKKFLKRRLLRLDE |

In particular embodiments, the targeting sequence is a cell penetrating peptide (CPP). Cell penetrating peptides may also be known as cell-permeable peptides or protein transduction domains (PTDs). CPPs/PTDs are a class of small peptides capable of penetrating the plasma membrane of mammalian cells (Lindgren et al., Trends Pharmacol. Sci. 21:99-103, 2000). Among the best-known PTDs are the HIV transcription factor TAT, the Antp peptide derived from the Drosophila melanogaster homeodomain protein, the herpes simplex virus protein VP22, and arginine oligomers (Schwarze & Dowdy, Trends Pharmacol. Sci. 21:45-48, 2000; Lundberg et al., Mol. Ther. 8:143-150, 2003; Snyder & Dowdy, Pharm. Res. 21:389-393, 2004; Johnson et al., J Invest Dermatol. 131(3): 753-761, 2011). PTDs are characterized by a high content of positively charged arginine and lysine amino acid residues, suggesting that the positive charge and the guanidinium group of arginine residues are essential to the transport (Zaro & Shen, Biochem. Biophys. Res. Commun. 307:241-247, 2003). These peptides have been reported to transport conjugated peptides, oligonucleotides, and even small particles such as liposomes across mammalian cells. In particular embodiments, the TAT peptide is an amino acid sequence set forth in SEQ ID NO: 2. Additional information regarding cell-penetrating peptides is provided for instance in: Derakhshankhah et al. (Biomed Pharmacother 108:1090-1096, 2018), Allen et al. (Biomolecules 8(3), July 11, 2018), Borrelli et al. (Molecules 23(2); Jan. 31, 2018), Kalafatovic et al. (Molecules 22(11), Nov. 8, 2017), Bechara et al. (FEBS Lett. 587(12):1693-1702, 2013), Yi (J Bacteriol Virol. 43(3):177-185, 2013), Tashima (Bioorganic & Med Chem Lett. 27(2):121-130, 2017). In particular embodiments, the CPPs are amino acid sequences selected from those listed in Table 2.

TABLE 2
Exemplary CPP Sequences

| | | SEQ |
|---|---|---|
| | | ID |
| Name | Sequence | NO: |
| Amphipathic | KLALKLALKALKAALKLA | 66 |
| peptide | | |
| HIV VP22 | DAATATRGRSAASRPTERPRAPARSASRPRRPVE | 67 |
| Hph-1 | YARVRRRGPRR | 68 |
| Inv3 | TKRRITPKDVIDVRSTVTTEINT | 69 |
| kFGF | AAVALLPAVLLALLAP | 70 |
| signal | | |
| peptide | | |
| M918 | MVTVLFRRLRIRRACGPPRVRV | 71 |
| MPG | GALFLGWLGAAGSTMGAPKKKRKV | 72 |
| Penetratin | RQIKIWFQNRRMKWKK | 73 |
| Pep-1 | KETWWETWWTEWSQPKKKRKV | 74 |
| pVEC | LLILRRRIRKQAHAHSK | 75 |
| Poly-Lysine | H-(K)ₙ-OH (n = 8 to 25) | N/A |
| Poly- | H-(R)ₙ-OH (n = 8 to 25) | N/A |
| Arginine | | |
| Pβ | GALFLGFLGAAGSTMGAWSQPKKKRKV | 76 |
| R6/W3 | RRWWRRWRR | 77 |
| SAP | VRLPPPVRLPPPVRLPPP | 78 |
| Syn B1 | RGGRLSYSRRRFSTSTGR | 79 |
| Syn B3 | RRLSYSRRRF | 80 |
| TAT | YGRKKRRQRRR | 2 |
| TAT | GRKKRRQRRRPPQ | 81 |
| (alter- | | |
| native) | | |
| TP10 | AGYLLGKINLKALAALAKKIL | 82 |
| Transportan | GWTLNSAGYLLKINLKALAALAKKIL | 83 |

Whether a polypeptide of the present disclosure is delivered to the appropriate organelle or cell can be determined by several methods. The polypeptide can be introduced into a cell as a composition including the polypeptide and a pharmaceutically acceptable carrier, preferably a liposome, phospholipid, or pH-activated lipid. Optimally, the carrier contains at least one metal ion donor, such as MnCl₂ and/or MgCl₂. Pharmaceutically acceptable carriers are described herein. Immunofluorescence analysis with an antibody that binds to the polypeptide can be used to determine whether the polypeptide has been delivered to a cell or to determine the intracellular distribution of the polypeptide after it is introduced. Alternatively, to determine whether the introduced polypeptide is targeted to the appropriate organelle of a cell, the appropriate organelle can be isolated, and the amount of the polypeptide in the organelle determined.

When determining whether a polypeptide of the disclosure is delivered to the appropriate organelle or cell, the polypeptide may be introduced to the cell as a polynucleotide encoding the polypeptide. The polypeptide is expressed from the polynucleotide and translated in the cytoplasm of the cell. The targeting of the polypeptide to an appropriate organelle, e.g. the nucleus, of a cell can be determined as described above. It should be noted that as used herein, a polynucleotide encoding the polypeptide can be used ex vivo to test whether a polypeptide is delivered to an appropriate organelle.

The term “polynucleotide” refers to a polymeric form of nucleotides of any length, either ribonucleotides or deoxynucleotides, and includes both double- and single-stranded DNA and RNA. A polynucleotide may include nucleotide sequences having different functions, including, for instance, coding sequences, and non-coding sequences such as regulatory sequences. Coding sequence, non-coding sequence, and regulatory sequence are defined below. A polynucleotide can be obtained directly from a natural source, or can be prepared with the aid of recombinant, enzymatic, or chemical techniques. A polynucleotide can be linear or circular in topology. For example, a polynucleotide can be a portion of a vector, such as an expression or cloning vector, or a fragment.

The term “recombinant” broadly describes various technologies whereby genes can be cloned, DNA can be sequenced, and protein products can be produced.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of polypeptide or polynucleotide sequences refers to sequences that are normally not part of a native polypeptide or polynucleotide found in and/or produced by a given yeast, bacterium, organism, microorganism, or cell.

Whether the polypeptide of the present disclosure retains UVDE activity once transported into the organelle can be determined by several methods. The polypeptide can be introduced to the cell as described herein, including introduction as a polypeptide and introduction as a polynucleotide that encodes the polypeptide. To measure activity after introduction to the cell, the appropriate organelle can be isolated, the polypeptide isolated from the organelle, and the activity of the isolated polypeptide determined. Alternatively, the repair rate of damaged DNA in the cell can be determined using, for instance, coding sequence-specific repair assays, photoproduct removal, and/or quantitative PCR.

Optionally, a polypeptide of the present disclosure further includes a series of consecutive amino acids encoding a domain or purification tag that facilitates the isolation, preferably purification, of the polypeptide. An “isolated” polypeptide or polynucleotide means a polypeptide or polynucleotide that has been either removed from its natural environment, produced using recombinant techniques, or chemically or enzymatically synthesized. Preferably, a polypeptide or polynucleotide of this disclosure is purified, i.e., essentially free from any other polypeptide or polynucleotide and associated cellular products or other impurities. For instance, domains or purification tags that are useful in the isolation of a polypeptide that has UVDE activity, include a histidine domain (which can be isolated using nickel-chelating resins), an S-peptide domain (which can be isolated using an S-protein, see Kim et al., Protein Sci 2:348-356, 1993), and a chitin binding domain (which can bind to chitin beads, see Chong et al., Gene, 192, 271-281, 1997 and Watanabe et al., J. Bacteriol., 176, 4465-4472, 1994). The domain or purification tag can be present at either the amino-terminal or carboxy terminal end of the polypeptide. In particular embodiments, the domain or purification tag can be cleaved from the remainder of the polypeptide (e.g., the polypeptide having UVDE activity linked to at least one targeting sequence) by the use of a protease or self-cleaving sequence. Optionally, in such embodiments the engineered polypeptide includes a sequence which governs binding of and cleavage by a protease. Following isolation of such a chimeric polypeptide, the cognate protease is used to cleave the polypeptide precisely at the junction with the protease recognition sequence and the UVDE-containing protein is purified with no tag or other extraneous sequences remaining.

By way of example, a SUMO (small ubiquitin-related modifier) (Boddy et al., Oncogene 13:971-982, 1996) domain can be included, which domain can be cleaved from the resultant expressed protein using the highly specific and active SUMO (ULP-1) protease. See FIG. 8C for a representative embodiment containing SUMO. Systems and methods for purification of SUMOlyated proteins are known; see, for instance, U.S. Patent Publications No. 2005/0069988, 2013/0017554; U.S. Pat. No. 7,910,364. Specifically contemplated embodiments provide a chimeric polypeptide in which the purification tag (for instance, a His6 tag) is at the N-terminus followed by a protease cleavage site (such as SUMO), and the UVDE protein to be expressed (for instance, UVDE-TAT). Utilizing an expression construct with sequences encoding these components, the recombinant polypeptide is synthesized in Escherichia coli and (following optimized expression) cell lysates are applied to an affinity matrix that preferentially binds the His6 tagged polypeptide. The captured chimeric polypeptide is then removed from the affinity matrix and treated with a protease that will cleave between its recognition sequence (for instance, at the C-terminal end of SUMO) and the desired expressed protein (e.g., UVDE-TAT). If the protease itself includes a 6xHis tag, this resultant solution is then reapplied to an Ni-2+ affinity matrix, thus binding the His6 tagged polypeptides (both the SUMO cleavage product and the protease itself) but allowing the target protein (e.g., UVDE-TAT) to flow through the matrix as a pure protein. The final product does not contain the amino acid sequences that were used as affinity purification tags. A representative UVDE-TAT expression construct containing a His6 tag and SUMO domain is provided in SEQ ID NOs: 84 and 85 (amino acid and nucleotide sequence, respectively); see also FIG. 8C.

The present disclosure also provides polynucleotides encoding a polypeptide of the present disclosure, i.e., a polypeptide having UVDE activity and at least one heterologous targeting sequence. A polynucleotide may include nucleotide sequences having different functions, including for instance coding sequences, and non-coding sequences such as regulatory sequences. “Coding sequence” and “coding region” are used interchangeably and refer to a polynucleotide that encodes a polypeptide and, when placed under the control of appropriate regulatory sequences expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

Polynucleotides encoding a polypeptide of the disclosure may be obtained from a yeast, for example, Schizosaccharomyces pombe. Methods for isolating a polynucleotide encoding a polypeptide of the disclosure employs standard cloning techniques known in the art (see, e.g., Sambrook et al, Molecular Cloning: A Laboratory Manual., Cold Spring Harbor Laboratory Press (1989) or Ausubel et al., (Eds.) Current Protocols in Molecular Biology, John Wiley & Sons, Inc. New York, N.Y. (1994)).

Examples of polynucleotides include those encoding the Schizosaccharomyces pombe uve1 (nucleotides 1160-2959 of GenBank Accession No. NM_001022085.2; SEQ ID NO: 15), Neurospora crassa UV endonuclease (nucleotides 148-2118 of GenBank Accession No. D11392.1) and Deinococcus radiodurans UV endonuclease (nucleotides 1-918 of GenBank Accession No. AB033747.1).

A “vector” is a nucleic acid molecule capable of transporting a nucleotide sequence into a cell. Vectors may be, e.g., viruses, phage, a DNA vector, a RNA vector, a viral vector, a bacterial vector, a plasmid vector, a cosmid vector, or an artificial chromosome vector. An “expression vector” is any type of vector that is capable of directing the expression of a nucleotide sequence (e.g., a therapeutic protein and/or interfering RNA (iRNA) encoded by one or more genes carried by the vector) when it is present in the appropriate environment.

Vectors and other carriers can include regulatory sequences to control the expression of nucleotide sequences (e.g., therapeutic proteins as disclosed herein or iRNA). These regulatory sequences can be eukaryotic or prokaryotic in nature. In particular embodiments, the regulatory sequence can result in the constitutive expression of the one or more nucleotide sequences upon entry of the carrier into the cell. Alternatively, the regulatory sequences can include inducible sequences. Inducible regulatory sequences are well known to those skilled in the art and are those sequences that require the presence of an additional inducing factor to result in expression of the one or more nucleotide sequences. Examples of suitable regulatory sequences include binding sites corresponding to tissue-specific transcription factors based on endogenous nuclear proteins, sequences that direct expression in a specific cell type, the lac operator, the tetracycline operator and the steroid hormone operator. Any inducible regulatory sequence known to those of skill in the art may be used.

In particular embodiments, the nucleotide sequence is stably integrated into the genome of a cell. In particular embodiments, the nucleotide sequence is stably integrated into the genome of a cell so that the nucleotide sequence is expressible by the cell and preferably heritable and expressible by its cell progeny. In particular embodiments, the nucleic acid is stably maintained in a cell as a separate, episomal segment.

In particular embodiments, inserted nucleotide sequences include genes encoding therapeutic proteins. Genes may include not only coding sequences but also non-coding regulatory regions such as promoters, enhancers, and termination regions. The term further can include all introns and other DNA sequences spliced from the mRNA transcript, along with variants resulting from alternative splice sites. Nucleic acid sequences encoding proteins can be DNA or RNA that directs the expression of protein or RNA. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein or RNA. The sequences can also include degenerate codons of the native sequence or sequences that may be introduced to provide codon preference. Thus, a gene refers to a unit of inheritance that occupies a specific locus on a chromosome and includes transcriptional and/or translational regulatory sequences and/or a coding region and/or non-translated sequences introns, 5′ and 3′ untranslated sequences). The term “gene” includes various sequence polymorphisms, mutations, and/or sequence variants. In particular embodiments, the sequence polymorphisms, mutations, and/or sequence variants do not affect the function of the encoded transcript. A coding sequence is any nucleotide sequence that contributes to the code for the product of a gene. A non-coding sequence thus refers to any nucleic acid sequence that does not contribute to the code for the product of a gene.

Particular embodiments include variants of nucleotide or protein sequences disclosed herein. Variants include sequences having one or more additions, deletions, stop positions, or substitutions, as compared to a reference sequence.

An amino acid substitution can be a conservative or a non-conservative substitution. A “conservative substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; VV).

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp.

Non-conservative substitutions include those that affect the function of a protein in a statistically-significant manner. Non-conservative substitutions include those in which (i) a hydrophilic residue (e.g. Ser or Thr) is substituted by a hydrophobic residue (e.g. Leu, Ile, Phe, Val, or Ala); (ii) a Cys or Pro is substituted by any other residue; (iii) a residue having an electropositive side chain (e.g. Lys, Arg, or His) is substituted by an electronegative residue (e.g. Gln or Asp); or (iv) a residue having a bulky side chain (e.g. Phe), is substituted by one not having a bulky side chain, (e.g. Gly). Additional information is found in Creighton (1984) Proteins, W.H. Freeman and Company.

Variants incorporating stop positions can be biologically active fragments. Biologically active fragments have 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence.

In particular embodiments, a nucleotide or protein sequence that has at least 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to a UVDE nucleotide or protein disclosed herein can be used.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, N.Y. (1988); Biocomputing: Informatics and Genome Projects (Smith, D. W., ed.) Academic Press, N.Y. (1994); Computer Analysis of Sequence Data, Part I (Griffin, A. M., and Griffin, H. G., eds.) Humana Press, N.J. (1994); Sequence Analysis in Molecular Biology (Von Heijne, G., ed.) Academic Press (1987); and Sequence Analysis Primer (Gribskov, M. and Devereux, J., eds.) Oxford University Press, N.Y. (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp, CABIOS, 1989; 5:151-153 with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul et al., J. Mol. Biol., 1990; 215:403-410; DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this disclosure it will be understood that where sequence analysis software is used for analysis, the results of the analysis are based on the “default values” of the program referenced. As used herein “default values” will mean any set of values or parameters which originally load with the software when first initialized.

The term “damaged base” and “damaged bases” refers to structural deviations in nucleoside-5′-monophosphates present in a eukaryotic cell's genomic DNA. One type of structural deviation is a covalent joining of the adjacent pyrimidines through the formation of a cyclobutane ring structure at the C5 and C6 positions. Another type of structural deviation is an imidazole ring fragmentation of a purine (either adenine or guanine). The location of such structural deviations in a cell's genomic DNA is referred to as a “lesion.” The term “genomic DNA” refers to the DNA present in the nucleus and the mitochondria of a cell. Damaged bases preferably arise from, for instance, UV radiation, ionizing radiation, oxidative stress, alkylation damage, and deamination. Examples of lesions include cis-syn and trans-syn II cyclobutane pyrimidine dimers, FapyA and FapyG (Lloyd, Mutat. Research, 408:159-170, 1998; and Lloyd, Prog Nucl Acid Res Mol Biol, 62:155-175, 1999).

Compositions. UVDE polypeptides and nucleotides encoding UVDE polypeptides disclosed herein can be formulated into compositions for administration to a subject. A UVDE composition includes UVDE polypeptides disclosed herein or nucleotides encoding UVDE polypeptides disclosed herein, or both. In particular embodiments, UVDE polypeptides include one or more targeting sequence(s), cell penetrating sequence(s), and/or purification tags. Compositions can advantageously include any pharmaceutically acceptable carriers which include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety and purity standards as required by United States FDA Office of Biological Standards and/or other relevant foreign regulatory agencies.

One or more metal ion donor(s) is included in embodiments of compositions which include a UVDE polypeptide. Such metal ions may be magnesium (e.g., 1-10 mM) or manganese (e.g., 0.1-1 mM), for instance. Such metal donor may be magnesium phosphate (e.g., (Mg(H₂PO₄)₂)xH₂O, (MgHPO₄)xH₂O, or (Mg₃(PO₄)₂)xH₂O), magnesium sulfate (e.g., MgSO₄(H₂O)ₓ where 0≤x≤7), magnesium chloride (e.g., MgCl₂(H₂O)ₓ, where 0≤x≤12), a manganese oxide (e.g., MnO, Mn₂O₃, Mn₃O₄, MnO₃, Mn₂O₇), manganese dioxide (e.g., MnO₂), manganese chloride, (MnCl₂(H₂O)ₓ, where X=0, 2, or 4), manganese sulfate (MnSO₄)(H₂O)ₓ, where x≤6), and so forth.

“Prodrugs” refer to compounds that can undergo biotransformation (e.g., either spontaneous or enzymatic) within a subject to release, or to convert (e.g., enzymatically, mechanically, electromagnetically, etc.) an active or more active form of the therapeutic after administration. Prodrugs can be used to overcome issues associated with stability, toxicity, lack of specificity, or limited bioavailability and often offer advantages related to solubility, tissue compatibility, and/or delayed release (See e.g., Bundgard, Design of Prodrugs, pp. 7-9, 21-24, Elsevier, Amsterdam, 1985; and Silverman, The Organic Chemistry of Drug Design and Drag Action, pp. 352-401, Academic Press, San Diego, Calif., 1992).

The compositions may further include pharmaceutically acceptable salts. Exemplary pharmaceutically acceptable salts include acetate, acid citrate, acid phosphate, ascorbate, benzenesulfonate, benzoate, besylate, bisulfate, bitartrate, bromide, chloride, citrate, ethanesulfonate, formate, fumarate, gentisinate, gluconate, glucaronate, glutamate, lactate, methanesulfonate, nitrate, iodide, isonicotinate, maleate, oleate, oxalate, p-toluenesulfonate, pamoate (i.e., 1,1′-methylene-bis-(2-hydroxy-3-naphthoate)), pantothenate, phosphate, saccharate, salicylate, succinate, sulfate, tannate and tartrate salts.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol and mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers and polysaccharides.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

In particular embodiments, compositions can include liposomes. Liposomes are self-assembling phospholipid bilayer structures that can be prepared from natural or synthetic phospholipid sources. These vesicles can encapsulate water soluble molecules in the aqueous volume while water insoluble molecules can be embedded in the hydrophobic region of the lipid bilayer. The simplest and the most widely used method for preparing liposomes is the thin lipid film hydration method introduced by Bangham et al. (J Mol Biol , 13:238, 1965). The constituents of a liposomal delivery system are the primary determinants of the preparation method to be employed. For instance, hydrophobic molecules can be included during the lipid film formation process (passive loading), whereas water soluble molecules can be introduced during the hydration step (passive loading) or incorporated later by active loading procedures using ion gradients. The phospholipid backbone of the liposomes includes saturated or unsaturated phospholipids with acyl chain length of 14 to 20 carbons. Surface modification by hydrophilic polymers is a commonly used method in liposomal delivery systems. The main goals of surface modification are prevention of particle aggregation and reduction of the capture of the liposomes by cells of the reticuloendothelial system. Due to their low degree of immunogenicity and antigenicity, polyethylene ethylene glycol (PEG) molecules of various chain lengths can be used to provide a protective shield over the phospholipid bilayer. PEG is a linear polyether diol that has a chemically inert backbone and hydroxyl groups available for derivatization. There are commercially available PEG derivatives that are covalently bound to phospholipids, functional groups, proteins, and even fluorescent probes. In certain embodiments, the liposomes contain at least one metal ion donor, such as MnCl₂ and/or MgCl₂; for instance, specific liposome embodiments contain 1 mM MnCl₂ and/or 10 mM MgCl₂.

In particular embodiments, for topical administration, the formulation can further include a penetration enhancer. The penetration enhancer can be a skin penetration enhancer. A skin penetration enhancer is a molecule that promotes the diffusion of polypeptides through the skin. A variety of compounds have been shown to be effective skin penetration enhancers. See, Percutaneous Penetration Enhancers (Smith et al., CRC Press, Inc., Boca Raton, Fla. 1995). Exemplary skin penetration enhancers include sulfoxides such as dimethylsulfoxide (DMSO) and decylmethylsulfoxide (CioMSO); ethers such as diethylene glycol monoethyl ether and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; the 1-substituted azacycloheptan-2-ones, particularly 1-n-dodecylcyclazacycloheptan-2-one; alcohols such as ethanol, propanol, octanol, benzyl alcohol, etc.; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; organic acids, particularly salicylic acid and salicylates, citric acid, and succinic acid; polyacrylic acids such as a carbomer (CARBOPOL™, B. F. Goodrich Company) and copolymers of C10 to C30 alkyl acrylates and one or more monomers of acrylic acid, methacrylic acid or one of their simple esters crosslinked with an allyl ether of sucrose or an allyl ether of pentaerythritol (PERMULEN™, B.F. Goodrich Company); galactomannan gums such as guar gum or locust bean gum; polysaccharide gum such as agar gum, alginate, carob gum, carrageen gum, ghatti gum, guar gum, karaya gum, kadaya gum, locust bean gum, rhamsan gum, xanthan gum, or a mixture thereof; and cellulose derivatives such as ethyl cellulose, methyl cellulose, hyrdoxypropyl cellulose, and mixtures thereof.

In particular embodiments, the compositions can be in the form of, e.g., gels, ointments, pastes, lotions, creams, sprays, foams, liquids, aerosol, suspension, emulsion, hydrogels, or powders. It is particularly contemplated that the compositions may be formulated as shampoos, soaps, body washes, and the like.

A gel is a substantially dilute cross-linked system, which exhibits no flow when in the steady-state. Most gels are liquid; however they behave more like solids due to the three-dimensional cross-linked network within the liquid. Gels can have properties ranging from soft and weak to hard and tough.

An ointment is a homogeneous, viscous, semi-solid preparation, most commonly a greasy, thick oil (oil 80%-water 20%) with a high viscosity. Ointments have a water number, which is the maximum quantity of water that 100 g of a base can contain at 20° C.

A paste includes three agents—oil, water, and powder, one of which includes a therapeutic agent. Pastes can be an ointment in which a powder is suspended.

A lotion also includes oil, water, and powder, but can have additional components (e.g., alcohol to hold the emulsion together) and often has a lower viscosity than a paste.

A cream is an emulsion of oil and water in equal proportions. Creams are thicker than lotions and maintain their shape when removed from a container.

Topical formulations disclosed herein can include components, such as animal and vegetable fats, oils, waxes, paraffins, starch, tragacanth, cellulose derivatives, polyethylene glycols, silicones, bentonites, silicic acid, talc, titanium oxide, and zinc oxide, or mixtures thereof. In particular embodiments, topical formulations may include thickening agents, surfactants, organic solvents, and/or tonicity modifiers. Optionally, in certain embodiments the topical formulations include one or more of retinol, tretinoin, vitamin A, vitamin C, hydroquinones, alpha hydroxy acids (AHAs), and/or beta hydroxy acids (BHAs).

In certain embodiments, skin tanning agents may be included in the composition. Common cosmetic ingredients for “artificial” tans (self-tanners) include for instance, dihydroxyacetone (DHA; encapsulated or not), erythrulose, and so forth. Additional compounds that may be used to alter skin color include: eicosanoids, retinoids, estrogens, melanocyte-stimulating hormone, endothelins, psoralens, hydantoin, forskolin, cholera toxin, isobutylmethylxanthine, diacylglycerol analogues, L-Tyrosine, and copper (available in many different chemical compounds). Optionally, one or more compounds that stimulate melanogenesis may be included, such as a SIK-inhibitor, an activator of melanocortin 1 (MC₁) receptor, α-melanocyte-stimulating hormone (α-MSH; Varga et al., J Mol. Neurosci 50(3)558-570, 2013; Schioth et al., Brit J Pharmacol 124(1):75-82, 1998)) or an analog thereof (such as afamelanotide, a.k.a. melanotan-I or Scenesse™; Hadley & Dorr, Peptides 27(4):921-930, 2006).

In particular embodiments, topical formulations can be prepared using thickening agents, such as carboxymethylcellulose sodium, sodium starch glycollate type C, or Carbomers such as Carbopol® (Lubrizol Advanced Materials, Inc. Cleveland, Ohio, USA) 934, 980, 981, 1382, 5984, or 2984. In particular embodiments, topical formulations can be prepared using surfactants, such as Pluronic® (BASF Corporation, Mount Olive, N.J., USA) co-polymers, such as Pluronic® F-127, and/or a Pluronic® co-polymer having the formula

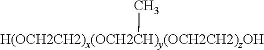

or H[OCH₂CH₂]₄₉[OCHCH₂]₆₇[OCH₂CH2]₄₉OH; propyl glycol, polypropylene glycol (PPG) stearyl ethers, such as PPG ethers of stearyl alcohol including PPG-20 methyl glucose ether distearate, PPG-15 Stearyl Ether, and PPG-11 Stearyl Ether.

In particular embodiments, topical formulations such as gel formulations may include an organic solvent (e.g. a lower alkyl alcohol, such as ethyl alcohol or isopropyl alcohol; a ketone, such as acetone or N-methyl pyrrolidone; a glycol, such as propylene glycol; or mixtures thereof) present in an amount of 1% to 99%. In particular embodiments, an organic solvent may be present in an amount of 60% to 80%. In particular embodiments, topical formulations may include a cellulose derivative, such as hydroxyl ethyl cellulose, hydroxy propyl cellulose, hydroxy propyl methyl cellulose, methyl cellulose, carboxy methyl cellulose, sodium carboxy methyl cellulose, or ethyl cellulose, or combinations thereof present in an amount of 0.1% to 20%. In particular embodiments a cellulose derivative may be present in an amount of 0.5% to 5%.

In particular embodiments, topical formulations such as gel formulations include any suitable tonicity modifier. Exemplary suitable tonicity modifiers include sodium chloride, potassium chloride, mannitol, sucrose, lactose, fructose, maltose, dextrose, dextrose anhydrous, propylene glycol, and glycerol. In particular embodiments, the tonicity modifier can be present in an amount of 0.5% to 1% by weight. In particular embodiments, a tonicity modifier can be present in an amount of 0.8% to 1% by weight of the topical formulation. In particular embodiments, buffers can be present in the topical formulations. Exemplary buffers include phosphate buffered saline (PBS) acetate buffers, such as sodium acetate trihydrate or glacial acetic acid; and citrate buffers, such as sodium citrate dihydrate and citric acid.

In particular embodiments, the compositions may include one or more polymeric surfactants. Polymers having surfactant properties (polymeric surfactant) can be hydrophobically modified polyacrylic acid (trade name Pemulen™ TR-1 and TR-2), water-soluble or water-swellable copolymers based on acrylamidoalkyl sulfonic acid and cyclic N-vinylcarboxamides (tradename Aristoflex® AVC), water-soluble or water-swellable copolymers based on acrylamidoalkyl sulfonic acid and hydrophobically modified methacrylic acid (tradename Aristoflex® HMB), and a homopolymer of acrylamidoalkyl sulfonic acid (tradename Granthix APP). Another class of notable polymeric emulsifier includes hydrophobically-modified, crosslinked, anionic acrylic copolymers, including random polymers, but may also exist in other forms such as block, star, graft, and the like.

In particular embodiments, the compositions can also include one or more moisturizing agents or an emollient component, for example mineral oil, dimethicone, cyclomethicone, cholesterol, hyaluronic acid, aloe Vera (or other plant-derived preparations or extracts), or combinations thereof. In some embodiments, the anhydrous composition includes liquid emollients such as polyhydric alcohols, polyols, saccharides, triglycerides, hydrocarbons, silicones, fatty acids, fatty, esters, fatty alcohols, and blends thereof. In some embodiments, the moisturizing agent is present from 0.5 wt % to 10 wt % of the total composition.

Compositions disclosed herein may contain preservatives to prevent the growth of harmful microorganisms. While it is in the aqueous phase that microorganisms tend to grow, microorganisms can also reside in the oil phase. As such, preservatives which have solubility in both water and oil are preferably employed in the present compositions. The traditional preservatives for cosmetics and pharmaceuticals are alkyl esters of para-hydroxybenzoic acid. Other preservatives which have more recently come into use include hydantoin derivatives, propionate salts, cationic surfactants such as benzalkonium chloride; benzyl alcohol, sorbic acid, and a variety of quaternary ammonium compounds.

In particular embodiments, topical formulations such as gel formulations may have a viscosity of at least 1,000 centipoise (cps). In particular embodiments, topical formulations such as gel formulations may have a viscosity of at least 3,000 cps. In particular embodiments, the viscosity of topical formulations will not exceed 50,000 cps.

Powders and sprays particularly may benefit from the inclusion of excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane. The compositions of the disclosure can be alternatively administered by aerosol. This is accomplished by preparing an aqueous aerosol, liposomal preparation, or solid particles containing a composition of the disclosure. A non-aqueous (e.g., fluorocarbon propellant) suspension also could be used. Sonic nebulizers can be preferred because they minimize exposing the compositions to shear, which can result in degradation of the composition.

In particular embodiments, a formulation disclosed herein includes a compound that delivers the active compound to the interior of cells, preferably to the interior of living skin cells under the skin's stratum corneum. Accordingly, such compounds deliver the active compounds across the stratum corneum and then across the outer cellular membrane of living cells. Examples of such compounds include liposomes, phospholipids, and pH-activated lipids (see, e.g., U.S. Pat. No. 5,190,762).

In particular embodiments, a formulation disclosed herein includes a sunscreen (sunblock) composition. A sunscreen can advantageously additionally include at least one further UVA filter and/or at least one further UVB filter and/or at least one inorganic pigment, preferably an inorganic micropigment. The UVB filters can be oil-soluble or water-soluble. Advantageous oil-soluble UVB filter substances are, for example: 3-benzylidenecamphor derivatives, preferably 3-(4-methylbenzylidene)camphor and 3-benzylidenecamphor; 4-aminobenzoic acid derivatives, preferably 2-ethylhexyl 4-(dimethylamino)benzoate and amyl 4-(dimethylamino)benzoate; esters of cinnamic acid, preferably 2-ethylhexyl 4-methoxycinnamate and isopentyl 4-methoxycinnamate; derivatives of benzophenone, preferably 2-hydroxy-4-methoxybenzophenone, 2-hydroxy-4-methoxy-4′-methylbenzophenone and 2,2′-dihydroxy-4-methoxybenzophenone; esters of benzalmalonic acid, preferably di(2-ethylhexyl)4-methoxybenzalmalonate. Advantageous water-soluble UVB filter substances are, for example: salts of 2-phenylbenzimidazole-5-sulphonic acid, such as its sodium, potassium or its triethanolammonium salt, and the sulphonic acid itself; sulphonic acid derivatives of benzophenones, preferably 2-hydroxy-4-methoxybenzophenone-5-sulphonic acid and salts thereof; sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl) benzenesulphonic acid and salts thereof. Sunscreen formulations optionally may include inorganic particulate compound(s) that reflect, scatter, and/or absorb UV light. For instance, titanium dioxide, zinc oxide, or a combination of both may be included. For additional discussion of sunscreen components, see the IARC Handbooks of Cancer Prevention, Vol. 5 (2001) “Sunscreens” (ISBN-13 978-92-82-3005-2; available online at publications.iarc.fr).

Additional embodiments provide delivery devices that are pre-loaded (or pre-wetted) with a composition that includes a UVDE polypeptide as provided herein. By way of example, such devices include wipes, towels and towelettes, sponges, cloths and so forth. Optionally, the delivery device may include a handle or extension, for instance to assist in application of the composition to skin of a subject (for instance, so an individual can apply the UVDE polypeptide-containing composition to their own back, scalp, or other difficulty to reach location). In some embodiments involving such a handle or extension, the therapeutic composition is contained in a pad or other removable (and replaceable) element.

Compositions can also be incorporated into wound dressings (e.g., bandages, adhesive bandages, transdermal patches), and more generally into devices (such as clothing, hats, masks, and so forth) intended to prevent or reduce wounding or damage that might otherwise be caused by exposure to solar and/or UV radiation. Generally, in wound dressing embodiments, compositions are embedded within puffs, gauzes, fleeces, gels, powders, sponges, or other materials that are associated with a second layer to form a wound dressing.

Absorption enhancers can also be used to increase the flux of the composition, and particularly the therapeutic protein within the composition, across the skin. The rate of such flux can be controlled by either providing a rate controlling membrane or dispersing the therapeutic protein in a polymer matrix or gel. In particular embodiments, compositions of the present invention can, for example, be applied to a plaster, patch, bandage, film, non-adhesive sheet silicone (for instance, to enhance scar reduction), polymer dressings, microsponges (Kaity et al., J Adv Pharm Technol Res. 1(3):283-290, 2010; Osmani et al., Saudi Pharma J. 23(5):562-572, 2015; Pawan & Prashant, Int J Pharma Sci Res. 7(7):2756-2761, 2016) and fabrics (including clothing). Specifically contemplated are embodiments in which the compositions are applied to an article of clothing (such as a shirt, mask, hat, etc.) which comes into direct contact with the skin of the person wearing the clothing. Alternatively, the therapeutic protein or a composition containing it can be applied to a protective insert that is brought and/or held in contact with the skin.

In particular embodiments, the second layer of a wound dressing can be, for example, an elastomeric layer, vapor-permeable film, waterproof film, or a woven or nonwoven fabric or mesh. The composition containing layer and second layer can be bonded using any suitable method (e.g., the application of adhesives, such as pressure sensitive adhesives, hot melt adhesives, curable adhesives; the application of heat or pressure, such as in lamination; a physical attachment through the use of stitching, studs, other fasteners).

Wound dressings may include adhesives for attachment to the skin or other tissue. Although any adhesive suitable for forming a bond with the skin or other tissue can be used, in particular embodiments a pressure sensitive adhesive is used. Pressure sensitive adhesives are generally defined as adhesives that adhere to a substrate when a light pressure is applied but leave little to no residue when removed. Pressure sensitive adhesives include solvent in solution adhesives, hot melt adhesives, aqueous emulsion adhesives, calenderable adhesives, and radiation curable adhesives.

The most commonly used elastomers in pressure sensitive adhesives include natural rubbers, styrene-butadiene latexes, polyisobutylene, butyl rubbers, acrylics, and silicones. In particular embodiments, acrylic polymer or silicone-based pressure sensitive adhesives can be used. Acrylic polymers can often have a low level of allergenicity, be cleanly removable from skin, possess a low odor, and exhibit low rates of mechanical and chemical irritation. Medical grade silicone pressure sensitive adhesives can be chosen for their biocompatibility.

Amongst the factors that influence the suitability of a pressure sensitive adhesive for use in wound dressings of particular embodiments is the absence of skin irritating components, sufficient cohesive strength such that the adhesive can be cleanly removed from the skin, ability to accommodate skin movement without excessive mechanical skin irritation, and good resistance to body fluids.

In particular embodiments, the pressure sensitive adhesive can include a butyl acrylate. While butyl acrylate pressure sensitive adhesives can generally be used for many applications, any pressure sensitive adhesive suitable for bonding skin can be used. Such pressure sensitive adhesives are well known in the art.

In particular embodiments, compositions of the present disclosure include UVDE polypeptides containing at least one heterologous targeting sequence encapsulated in liposomes in a hydrogel solution. In particular embodiments, the liposomes include a 2:2:5:1 molar ratio of 1,2-dioleoyl-sn-glycero-3-phosphoethanolamine (DOPE): 1,2-dioleoyl-sn-glycero-3-phosphocholine (DOPC): cholesteryl hemisuccinate (CHEMS): oleic acid. In certain embodiments, the liposomes contain at least one metal ion donor, such as MnCl₂ and/or MgCl₂. 1 mM; in specific examples, the liposomes contain 1 mM MnCl₂ and/or 10 mM MgCl₂. In particular embodiments, the hydrogel is included of Carbomer 940 in PBS. In particular embodiments, the final hydrogel concentration is 0.75% w/v. Example hydrogels include hyaluronic acid, aloe vera, or a combination thereof.

In particular embodiments, the compositions can be in the form of hydrogels. Hydrogels are typically prepared by cross-linking various monomers and/or polymers to provide a three-dimensional polymer network. Non-limiting examples of polymers include, polyoxyethylene-polypropylene block copolymers, ionic polysaccharides, such as chitosan or sodium alginate, cellulose, and biodegradable polymers, such as poly-lactides (PLA) and polyglycolides (PGA), butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG).

In particular embodiments, the compositions may be in the form of emulsions. An emulsion is a dispersed system containing at least two immiscible liquid phases, one of which is dispersed in the form of small droplets throughout the other, and an emulsifying agent in order to improve the stability of the system. There are two types of emulsions depending on the droplet size of the liquids present in the emulsions: macroemulsions and microemulsions. Light does not pass through macroemulsions because the droplets have average diameters of 10 to 1000 μm. These emulsions typically appear milky white. Microemulsions are stable systems having droplets which are significantly smaller, being 500 nm or smaller in diameter on the average. As such, microemulsions are translucent, and routinely transparent, in appearance. Microemulsions are an extraordinary type of emulsion that form spontaneously. Products having these systems are valued for their stability and small particle size, thus affording microemulsions a special consideration in the market place.

Compositions can also be depot preparations. Such long acting compositions may be administered by, for example, implantation (for example, subcutaneously). Thus, for example, compounds can be formulated with suitable polymeric or hydrophobic materials (for example as an emulsion in an acceptable oil) or ion exchange resins, or as sparingly soluble derivatives, for example, as sparingly soluble salts. Optionally, such preparations may include one or more injectable fillers.

Additionally, compositions can be delivered using sustained-release systems, such as semipermeable matrices of solid polymers containing at least one compound disclosed herein. Various sustained-release materials have been established and are well known by those of ordinary skill in the art. Sustained-release capsules may, depending on their chemical nature, release the compound following administration for a few weeks up to over 100 days.

Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with therapeutic compositions disclosed herein. Treating subjects includes delivering therapeutically effective amounts. Therapeutically effective amounts include those that provide effective amounts, prophylactic treatments and/or therapeutic treatments.

In particular embodiments, subjects are treated with UVDE compositions disclosed herein to prevent or treat skin disorders, melanoma, actinic keratosis, or non-melanoma skin cancer (NMSC). NMSCs can include basal cell carcinoma (BCC), squamous cell carcinoma (SCC), Merkel cell carcinoma, cutaneous lymphoma, Kaposi sarcoma, skin adnexal tumors, and sarcoma.

An actinic keratosis (AK), also known as a solar keratosis, is a crusty, scaly growth caused by damage from exposure to ultraviolet (UV) radiation. AK is considered a precancer because if left alone, it could develop into a skin cancer, most often the second most common form of the disease, squamous cell carcinoma (SCC). The most common type of precancerous skin lesion, AKs appear on skin that has been frequently exposed to the sun or to artificial sources of UV light, such as tanning machines. In rare instances, extensive exposure to X-rays can cause them. Above all, they appear on sun-exposed areas such as the face, bald scalp, ears, shoulders, neck and the back of the hands and forearms. They can also appear on the shins and other parts of the legs. They are often elevated, rough in texture and resemble warts. Most become red, but some are light or dark tan, white, pink and/or flesh-toned. They can also be a combination of these colors. In the beginning, AKs are frequently so small that they are recognized by touch rather than sight. Patients may have many times more invisible (subclinical) lesions than those appearing on the surface. Most often, actinic keratoses develop slowly and reach a size from an eighth to a quarter of an inch. Early on, they may disappear only to reappear later. Occasionally they itch or produce a pricking or tender sensation. They can also become inflamed and surrounded by redness. In rare instances, AKs can even bleed.

Basal cell carcinomas (BCCs) are abnormal, uncontrolled growths or lesions that arise in the skin's basal cells, which line the deepest layer of the epidermis (the outermost layer of the skin). BCCs often look like open sores, red patches, pink growths, shiny bumps, or scars and are usually caused by a combination of cumulative and intense, occasional sun exposure. BCC almost never spreads (metastasizes) beyond the original tumor site. More than 4 million cases of basal cell carcinoma are diagnosed in the U.S. each year. In fact, BCC is the most frequently occurring form of all cancers. More than one out of every three new cancers is a skin cancer, and the vast majority are BCCs.

Squamous cell carcinoma (SCC) is an uncontrolled growth of abnormal cells arising in the squamous cells, which compose most of the skin's upper layers (the epidermis). SCCs often look like scaly red patches, open sores, elevated growths with a central depression, or warts; they may crust or bleed. They can become disfiguring and sometimes deadly if allowed to grow. More than 1 million cases of squamous cell carcinoma are diagnosed each year in the U.S., and as many as 8,800 people die from the disease. Incidence of the disease has increased up to 200 percent in the past three decades in the U.S. SCC is mainly caused by cumulative ultraviolet (UV) exposure over the course of a lifetime; daily year-round exposure to the sun's UV light, intense exposure in the summer months, and the UV produced by tanning beds all add to the damage that can lead to SCC. SCCs may occur on all areas of the body including the mucous membranes and genitals, but are most common in areas frequently exposed to the sun, such as the rim of the ear, lower lip, face, balding scalp, neck, hands, arms and legs. Often the skin in these areas reveals telltale signs of sun damage, including wrinkles, pigment changes, freckles, “age spots,” loss of elasticity, and broken blood vessels.

Melanoma is the most dangerous form of skin cancer. The tumors originate in the pigment-producing melanocytes in the basal layer of the epidermis. Melanomas often resemble moles; some develop from moles. The majority of melanomas are black or brown, but they can also be skin-colored, pink, red, purple, blue or white. Melanoma is caused mainly by intense, occasional UV exposure (frequently leading to sunburn), especially in those who are genetically predisposed to the disease. Melanoma kills an estimated 10,130 people in the US annually. If melanoma is not recognized and treated early, the cancer can advance and spread to other parts of the body, where it becomes hard to treat and can be fatal. While it is not the most common of the skin cancers, it causes the most deaths.

In particular embodiments, a therapeutically effective amount of an UVDE composition disclosed herein is used to treat xeroderma pigmentosum (XP) patients. XP is an inherited condition characterized by an extreme sensitivity to UV rays from sunlight. This condition mostly affects the eyes and areas of skin exposed to the sun. Some affected individuals also have problems involving the nervous system. People with XP have a greatly increased risk of developing skin cancer. Most people with XP develop multiple skin cancers during their lifetime. These cancers occur most often on the face, lips, and eyelids. Cancer can also develop on the scalp, in the eyes, and on the tip of the tongue. In addition to an increased risk of eye cancer, XP is associated with noncancerous growths on the eye. Many of these eye abnormalities can impair vision. Studies suggest that people with XP may also have an increased risk of other types of cancer, including brain tumors. Thirty percent of people with XP develop progressive neurological abnormalities in addition to problems involving the skin and eyes. These abnormalities can include hearing loss, poor coordination, difficulty walking, movement problems, loss of intellectual function, difficulty swallowing and talking, and seizures. When these neurological problems occur, they tend to worsen with time. Researchers have identified at least eight inherited forms of XP: complementation group A (XP-A) through complementation group G (XP-G) plus a variant type (XP-V). The types are distinguished by their genetic cause. All of the types increase skin cancer risk, although some are more likely than others to be associated with neurological abnormalities.

In particular embodiments, a therapeutically effective amount of an UVDE composition disclosed herein is used to treat organ transplant patients. Transplant patients are given drugs such as cyclosporine and azathioprine to suppress their immune system so that it will not attack the donated organ as a foreign invader; the drugs enable the body to accept the organ. Unfortunately, immune-suppressed people, including recipients of all major solid organs (heart, lung, kidney, pancreas, liver), have a much higher risk of skin cancers than people in the general population. SCC is the most frequent problem, occurring 65 to 250 times more often in transplant patients, but melanoma also occurs 6 to 8 times more frequently. Kaposi's sarcoma, BCC, and Merkel cell carcinoma (a virulent but normally very rare skin cancer) are also more common in transplant patients.

In particular embodiments, the UVDE compositions disclosed herein can be used to treat skin disorders. Examples of skin disorders that may be treated by the compositions include sun burn, sun poisoning, plantar hyperkeratosis, blisters, tuberous sclerosis, seborrheic keratosis, keratosis pilaris, epidermolysis bullosa, multiple minute digitate hyperkeratosis, hyperkeratosis lenticularis perstans, stasis dermatitis, focal acral hyperkeratosis, follicular hyperkeratosis, lichenoid keratoses (lichen planus, lichen sclerosus), actinic lichenoid leukomelanoderma, Conradi-Eltinermann, epidermolytic ichthyosis, erythrokeratoderma variabilis, ichthyosis hystrix, KID syndrome, Netherton syndrome, Olmsted syndrome, Refsum disease, Sjogren-Larsson Syndrome, actinic keratosis, pachyonychia congenita, hyperhidrosis, warts, calluses, dermatitis (contact dermatitis, drug-induced dermatitis, allergic dermatitis, nummular dermatitis, perioral dermatitis, neurodermatitis, seborrheic dermatitis, and atopic dermatitis), psoriasis, acne, carbunculosis, cellulitis, furunculosis, granuloma, acanthosis nigricans, athlete's foot, bacterial vaginosis, balanitis, dermatofibrosarcoma protruberans, basal cell carcinoma, squamous cell carcinoma, melanoma, merkel cell carcinoma, keloid, cystic lymphangioma, Cavernous lymphangioma, venous malformation, epidermal nevi, bromhidrosis, dermatophytosis, candidiasis, onychomycosis, tinea (tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito), eczema, dyshydrotic eczema, decubitous ulcer, ecthyma, erysipalus, erythema multiforme, impetigo, insect bites, genital warts, hemangioma, herpes, hives, hyperhidrosis, filariasis, lentigines, lupus, miliaria, milkers nodules, molluscum contagiosum, myiasis, scabies, cutaneous larva migrans, furuncular myiasis, migratory myiasis, pediculosis, nevus araneus, panniculitis, paronychia, pemphigoid, photodermatitis (including various types of photosensitive dermatitis associated with HIV infection), pityriasis, pruritis vulvae, rosacea, trichomoniasis, vaginal yeast infection, vitiligo, xeroderma, angiofibroma, Bannayan-Riley-Ruvalcaba syndrome, basal cell nevus syndrome, Birt-Hogg-Dube syndrome, Blue rubber bleb nevus syndrome, Cowden disease, cutaneous t-cell lymphoma, diffuse microcystic lymphatic malformations, epidermolysis bullosa simplex, extramammary paget, familial multiple discoid fibromas, Hailey-Hailey disease, infantile hemangiomas, juvenile polyposis syndrome, Kaposi sarcoma, Kaposiform hemangioendothelioma, Keloid scar disease, Lhermitte-Duclos syndrome, metastatic melanoma, Muir-Torre syndrome, neurofibromatosis, nonmelanoma skin cancer, oral graft-versus-host disease, Pemphigus vulgaris, Peutz-Jeghers syndrome, Port-wine stains, Proteus syndrome, Proteus-like Syndrome, refractory hemangioendotheliomas in Maffucci syndrome, Sturge-weber syndrome, xeroderma pigmentosum, Hermansky-Pudlak syndrome, albinism, oculocutaneous albinism (OCA), Chediak-Higashi syndrome, Cockayne's syndrome (Neill-Dingwall syndrome), condition(s) customarily treated by a trichologist, photosensitivity disorder(s) associated with HIV (see, e.g., Koch, South Afr J HIV Med 18(1):676, 2017). and combinations thereof.

In particular embodiments, the skin disorder that is treated is angiofibroma. In particular embodiments, the skin disorder that is treated is pachyonychia congenita. In particular embodiments, a symptom of pachyonychia congenita selected from pain, itch or a combination thereof, is decreased upon treatment with the polypeptides and polynucleotides of the present disclosure. In particular embodiments, the skin disorder that is treated is xeroderma pigmentosum.

An “effective amount” is the amount of a compound necessary to result in a desired physiological change in the subject. Effective amounts are often administered for research purposes. Effective amounts disclosed herein can cause a statistically-significant effect in an animal model or in vitro assay relevant to the assessment of: repair of UV-induced DNA damage (e.g., removal of dipyrimidine photoproducts); number of tumors, size of tumors and/or total tumor burden caused by UV irradiation; frequency of or time to onset of UV-induced skin cancers; risk of death (hazard ratios) and/or increase in survival in subjects exposed to UV irradiation; amount of circulating inflammatory markers; and amount of circulating lymphocytes, monocytes and eosinophils.

A “prophylactic treatment” includes a treatment administered to a subject who does not display signs or symptoms of a UV-induced skin disorder or UV-induced immunological response, or displays only early signs or symptoms of a UV-induced skin disorder or UV-induced immunological response such that treatment is administered for the purpose of diminishing or decreasing the risk of developing a UV-induced skin disorder or UV-induced immunological response further. Thus, a prophylactic treatment functions as a preventative treatment against a UV-induced skin disorder or UV-induced immunological response. A UV-induced immunological response can include: an elevation in circulating lymphocytes, monocytes, and/or eosinophils; an elevation in circulating inflammatory cytokines and proteins; immune suppression following DNA damage. In particular embodiments, prophylactic treatments reduce, delay, or prevent: number of tumors, size of tumors and/or total tumor burden caused by UV irradiation; frequency of or time to onset of UV-induced skin cancers; risk of death (hazard ratios) in subjects exposed to UV irradiation; an increase in the amount of circulating inflammatory markers; an increase in the amount of circulating lymphocytes, monocytes and eosinophils; and/or immune suppression caused by UV-induced DNA lesions.

As one example of a prophylactic treatment, a topical formulation disclosed herein can be administered to a subject who is at risk of developing skin cancer. An effective prophylactic treatment of skin cancer can occur when time to onset of skin cancer is delayed or prevented, when an increase in repair of damaged DNA occurs, when the frequency of actinic keratosis (AK) is reduced or prevented, or when the frequency of skin carcinomas is reduced or prevented.

As another example of a prophylactic treatment, a topical formulation disclosed herein can be administered to a subject who is at risk of developing a UV-induced inflammatory response. An effective prophylactic treatment of the UV-induced inflammatory response can occur when an increase in circulating inflammatory cytokines or proteins are reduced or prevented; when an increase in circulating lymphocytes, monocytes, and/or eosinophils are reduced or prevented; or when immune suppression is minimized or prevented.

A “therapeutic treatment” includes a treatment administered to a subject who displays symptoms or signs of a UV-induced skin disorder or UV-induced immunological response and is administered to the subject for the purpose of diminishing or eliminating those signs or symptoms of a UV-induced skin disorder or UV-induced immunological response. The therapeutic treatment can reduce, control, or eliminate the occurrence of a UV-induced skin disorder or UV-induced immunological response and/or reduce, control or eliminate side effects of a UV-induced skin disorder or UV-induced immunological response. In particular embodiments, therapeutic treatments reduce, delay, or prevent time to onset of skin cancer, UV-induced DNA damage, frequency of actinic keratosis, and/or frequency of skin carcinomas.

As one example of a therapeutic treatment, a topical formulation disclosed herein can be administered to a subject who is at risk of developing skin cancer. An effective prophylactic treatment of skin cancer occurs when time to onset of skin cancer is delayed or prevented, when an increase in repair of damaged DNA occurs, when the frequency of actinic keratosis (AK) is reduced or prevented, or when the frequency of skin carcinomas is reduced or prevented.

As another example of a therapeutic treatment, a topical formulation disclosed herein can be administered to a subject who is at risk of developing a UV-induced inflammatory response. An effective prophylactic treatment of the UV-induced inflammatory response can occur when an increase in circulating inflammatory cytokines or proteins are reduced or prevented; when an increase in circulating lymphocytes, monocytes, and/or eosinophils are reduced or prevented; or when immune suppression is minimized, controlled, or prevented.

In the context of a UV-induced skin disorder or UV-induced immunological response, therapeutically effective amounts can: decrease the number of tumors, size of tumors and/or total tumor burden caused by UV irradiation; reduce frequency of or delay time to onset of UV-induced skin disorders; decrease risk of death (hazard ratios) in subjects exposed to UV irradiation; reduce or prevent the amount of circulating inflammatory cytokines or proteins caused by UV-induced inflammatory response; reduce or prevent the amount of circulating lymphocytes, monocytes and eosinophils caused by UV-induced inflammatory response; and/or reduce or prevent immune suppression caused by UV-induced DNA lesions.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

For administration, therapeutically effective amounts (also referred to herein as doses) can be initially estimated based on results from in vitro assays and/or animal model studies. The actual dose amount administered to a particular subject can be determined by a physician, veterinarian or researcher taking into account parameters such as physical and physiological factors including target, body weight, severity of skin cancer, type of skin cancer, stage of skin cancer, previous or concurrent therapeutic interventions, idiopathy of the subject and route of administration.

Useful doses of UVDE polypeptides and nucleotides can often range from 0.1 to 5 μg or from 0.5 to 1 μg. In other examples, a dose can include 1 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 0.1 to 5 mg or from 0.5 to 1 mg. In other examples, a dose can include 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more.

Useful concentrations of the UVDE recombinant polypeptides encapsulated in liposomes disclosed herein can range from 0.5 μg/mL to 100 μg/mL. In particular embodiments, the concentrations can range from 1 μg/mL to 50 μg/mL. In particular embodiments, the concentrations of the recombinant polypeptides can include 0.5 μg/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, or more. In certain embodiments, the liposomes contain 1 mM MnCl₂ and/or 10 mM MgCl₂.

In particular embodiments, UVDE polypeptides or nucleotides may be present from 0.1 wt. % to 10 wt. %, 0.1 wt. % to 9 wt. %, 0.1 wt. % to 8 wt. %, 0.1 wt. % to 7 wt. %, 0.1 wt. to 6 wt. %, 0.1 wt. % to 5 wt. %, 0.1 wt. % to 4 wt. %, 0.1 wt. % to 3 wt. %, 0.1 wt. % to 2 wt. %, or 0.1 wt. % to 1 wt. %. Specific examples include 0.1 wt. %, 0.5 wt. %, 1 wt. %, 2 wt. %, 5 wt. %, 10 wt. %, and ranges between any two of these values. The weight percentages disclosed herein may be weight-to-weight or weight -to-volume percentages with respect to the total amount of the composition.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, or weekly.

The compositions described herein can be administered by, for example, injection or ingestion. Routes of administration can include intradermal, topical, oral, and/or subcutaneous injection. In particular embodiments, the compositions disclosed herein can be formulated for topical administration.

In particular embodiments, administration of the UVDE composition is by topical application, transdermal, percutaneous, or microneedle injection. Administration can also be, for example, parenteral, subcutaneous, intravenous, intramuscular, intraperitoneal, transdermal, oral, buccal, or ocular routes, or intravaginally, by inhalation, by depot injections, or by implants.

In particular embodiments, the UVDE polypeptide or nucleotide compositions are administered percutaneously, and the polypeptide/nucleotide reaches epidermal and dermal layer through percutaneous absorption. In some embodiments, the percutaneous application of the anhydrous composition does not result in systemic absorption. In some embodiments, percutaneous delivery is aided by the use of ultrasound technology. The ultrasound energy is applied to percutaneous delivery composition over the tissue and assists the diffusion of the composition past the tissue. Also contemplated are delivery methods involving iontophoresis, electroporation, magnetophoresis, laser assisted peptide delivery, and so forth.

Also contemplated are embodiments in which the UVDE polypeptide is provided along with one or more protease inhibitor(s), either concurrently (in the same or a different delivery composition) or sequentially. In certain embodiments, a protease inhibitor may be used to stabilize therapeutic proteins or peptides in a composition as described herein, for instance by inhibiting degradation of the therapeutic protein or peptide. The outer layer of the skin, the stratum corneum (SC), contains an array of proteases capable of degrading proteins and peptides. Thus, proteases located in the SC may constitute a barrier to achieving the full therapeutic benefits of topical skin applications. Not only may a protease inhibitor act to prolong therapeutic activity by increasing peptide half-life, but the inhibitor also may reduce or prevent the production of pro-inflammatory fragments from native skin proteins. A peptide or protein used in combination with an appropriate protease inhibitor can exhibit a greater half-life. Therefore, such the therapeutic protein or peptide need not be supplied at the higher levels required when the peptide is used in the absence of protease inhibitor. A protease inhibitor can be selected to specifically target proteases that would be expected to degrade the selected bioactive peptide (e.g., a truncated UVDE); such a selection would be determined based on the length and/or sequence of the bioactive peptide. However, protease inhibitors need not necessarily be selected in any specific manner; for example, a protease inhibitor cocktail, which contains two or more inhibitors, can be employed. The following types of protease inhibitors can be incorporated compositions: serine protease inhibitors, cysteine protease inhibitors, aspartate protease inhibitors, metalloproteinase inhibitors, thiol protease inhibitors and threonine protease inhibitors.

Protease inhibitors are well known in the art. Non-limiting examples of protease inhibitors that can be incorporated in topical compositions include acetyl-pepstatin, AEBSF (4-[2-Aminoethyl] benzenesulfonyl fluoride) hydrochloride, ALLM (N-Acetyl-Leu-Leu-Met), ALLN (N-Acetyl-Leu-Leu-Nle-CHO), amastatin (Streptomyces sp.), ε-amino-n-caproic acid, aminopeptidase N inhibitor, α₁-antichymotrypsin, antipain (hydrochloride or dihydrochloride), α2-antiplasmin, antithrombin III, α1-antitrypsin, p-APMSF hydrochloride, aprotinin (e.g., from bovine lung), ATBI (an 11-residue peptide), benzamidine hydrochloride, bestatin, bestatin methyl ester, calpastatin, calpeptin, carboxypeptidase inhibitor, caspase inhibitor, cathepsin B inhibitor II, cathepsin G inhibitor I, cathepsin inhibitor II, cathepsin inhibitor III, cathepsin inhibitor I, cathepsin K inhibitor I, cathepsin K inhibitor II, cathepsin K inhibitor III, cathepsin L inhibitor I, cathepsin L inhibitor II, cathepsin L inhibitor IV, cathepsin L inhibitor V, cathepsin L inhibitor VI, cathepsin S inhibitor, cathepsin/subtilisin inhibitor, chymostatin, chymotrypsin inhibitor I, cystatin, 1,5-dansyl-glu-gly-arg chloromethyl ketone dihydrochloride, 3,4-dichloroisocoumarin, diisopropylfluorophosphate, dipeptidylpeptidase II inhibitor, dipeptidylpeptidase IV inhibitor I, dipeptidylpeptidase IV inhibitor II, E-64 protease inhibitor, ecotin, elastase inhibitor I, elastase inhibitor II, elastase inhibitor III, elastatinal, 6-amidino-2-naphthyl-4-guanidinobenzoate dimethanesulfonate, glu-gly-arg-chloromethyl ketone, 2-guanidinoethylmercaptosuccinic acid, hexadecylsulfonyl fluoride, .alpha.-iodoacetamide, kininogen, leuhistin, leupeptin hemisulfate, α₂-macroglobulin, DL-2-mercaptomethyl-3-guanidinoethylthiopropanoic acid, pepstatin A, phenylmethylsulfonyl fluoride, phosphoramidon Disodium Salt, PPack II trifluoroacetate salt, PPack dihydrochloride, prolyl endopeptidase inhibitor II, Na-tosyl-lys chloromethyl ketone hydrochloride, Na-tosyl-phe chloromethyl ketone, tripeptidylpeptidase II inhibitor, trypsin inhibitor (from corn or soybean), D-val-phe-lys chloromethyl ketone dihydrochloride, 1,3-di-(N-carboxybenzoyl-L-leucyl-L-leucyl)amino acetone, o-phenanthroline, ursolic acid (e.g., Rosemary extract), tranexamic acid (4-[aminomethyl]cyclohexane-1-carboxylic acid) (clinically marketed as Cyklokapron™ in the U.S. and as Transamin™ in Asia), Fmoc-Lys(Boc), Fmoc-Arg(Pmc), benzoyl-Arg-nitroanilide, benzoyl-Arg-naphthylamide, and α2-macroglobuline.

Chelators such as EDTA disodium salt dihydrate, EDTA tetrasodium salt, and EGTA are also recognized protease inhibitors; however, in the current instance they may inhibit the optimal function of the UVDE polypeptide. Thus there are specifically contemplated herein embodiments in which a therapeutic composition (such as a topical composition) including UVDE does not include a metal chelator. Alternatively, embodiments are contemplated wherein the UVDE and any chelating agent (no matter its purpose in the composition) are sequestered from each other, for instance by one or the other component being contained in a liposome or other delivery system. See, for instance, Song et al. (Int. J Nanomed. 9:3611-3621, 2014), which describes the application of EDTA in drug delivery systems involving liposomes.

The protease inhibitor may itself be a peptide or protein, such as an enzyme. Non-limiting examples of such inhibitors are the serpins, which include alpha-1-antitrypsin, complement 1-inhibitor, antithrombin, alpha-1-antichymotrypsin, plasminogen activator inhibitor 1, neuroserpin, and TIMP-1 (Yokose et al., J Invest Dermatol. 132:2800-2809, 2012).

Kits. Active component(s), including particularly at least one UVDE polypeptide, can be provided as kits. Kits can include one or more containers including one or more or more compounds as described herein, optionally along with one or more agents for use in therapy. For instance, some kits will include an amount of at least one sunscreen component, or at least one anti-inflammatory component.

Any active component in a kit may be provided in premeasured dosages, though this is not required; and it is anticipated that certain kits will include more than one dose.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as applicators, ampules, sponges, sterile adhesive strips, Chloraprep, gloves, and the like. Variations in contents of any of the kits described herein can be made. The instructions of the kit will direct use of the active ingredient(s) included in that kit to effectuate a clinical and/or therapeutic use described herein.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### First Set of Exemplary Embodiments

1. A recombinant polypeptide including: the truncated UV damage endonuclease (UVDE) shown in amino acids 229 to 599 of SEQ ID NO: 16, and the transactivator of transcription (TAT) amino acid sequence of SEQ ID NO: 2.

2. A recombinant polypeptide including: a truncated UV damage endonuclease (UVDE), wherein the truncation is amino-terminal (N-terminal) to a conserved region of the UVDE required for enzymatic activity, and at least one heterologous targeting sequence.

3. The recombinant polypeptide of embodiment 1, wherein the at least one heterologous targeting sequence is at the carboxy-terminus (C-terminus) of the UVDE or at the amino-terminus (N-terminus) of UVDE.

4. The recombinant polypeptide of embodiment 1, wherein the truncated UVDE is Schizosaccharoroyces pombe Uve1p lacking the first 228 amino acids of SEQ ID NO: 16,

5. The recombinant polypeptide of embodiment 2 or 3, wherein the at least one heterologous targeting sequence includes: a cell penetrating peptide; or a nuclear localization signal (NLS); or a NLS and a TAT protein transduction domain.

6. The recombinant polypeptide of embodiment 1, 2, or 3, wherein the recombinant polypeptide includes amino acids 1-383 of SEQ ID NO: 12 or amino acids 1-391 of SEQ ID NO: 14.

7. The recombinant polypeptide of embodiment 1, 2, or 3, wherein the recombinant polypeptide further includes one or more purification tags.

8. The recombinant polypeptide of embodiment 1, 2, or 3, wherein the recombinant polypeptide further includes at least one sequence that is specifically recognized by a protease, and which protease-recognition sequence is positioned between the truncated UVDE and the at least one heterologous targeting sequence.

9. The recombinant polypeptide of any one of the proceeding embodiments, wherein the recombinant polypeptide is encapsulated in a liposome.

10. A recombinant polynucleotide encoding: a truncated UV damage endonuclease (UVDE) sequence, wherein the truncation is 5′ to a conserved region of the UVDE sequence required for enzymatic activity; and at least one heterologous targeting sequence.

11. The recombinant polynucleotide of embodiment 10, wherein the heterologous targeting sequence is at the 3′ end of the UVDE sequence.

12. The recombinant polynucleotide of embodiment 10, wherein the truncated UVDE sequence is Schizosaccharomyces pombe Uvel lacking the first 684 nucleotides of SEQ ID NO: 15.

13. The recombinant polynucleotide of embodiment 10 or 12, wherein the at least one heterologous targeting sequence includes: a cell penetrating peptide sequence; a nuclear localization signal (NLS) sequence; or a NLS sequence and a TAT cell penetrating peptide sequence,

14. The recombinant polynucleotide of embodiment 10 or 12, wherein the recombinant polynucleotide includes nucleotides 1-1149 of SEQ ID NO: 11 or nucleotides 1-1173 of SEQ ID NO: 13.

16. The recombinant polynucleotide of embodiment 10 or 12, wherein the recombinant polynucleotide further includes one or more purification tag sequences.

16. The recombinant polynucleotide of embodiment 10 or 12, wherein the recombinant polynucleotide further includes at least one sequence that encodes a peptide sequence specifically recognized by a protease, and which protease-recognition sequence encoding sequence is positioned between the sequence encoding the truncated UVDE and the sequence encoding the at least one heterologous targeting sequence.

17. A vector including the polynucleotide of any one of embodiments 10-16.

18. A host cell including the recombinant polypeptide of embodiment 1, 2, or 3 or the vector of embodiment 17

19. A topical formulation including a therapeutically effective amount of the recombinant polypeptide of any one of embodiments 19 or the recombinant nucleotide of any one of embodiments 10-16.

20. The topical formulation of embodiment 19, wherein the topical formulation is incorporated in a lotion, a cream, an ointment, a paste, a powder, a sunscreen, a liquid, an aerosol, a suspension, an emulsion, a foam, a gel, a hydrogel, a plaster, a patch, a bandage, a wipe, a microsponge, an elastomer, or a film.

21. The topical formulation of embodiment 19, wherein the recombinant polypeptide or the recombinant polynucleotide is encapsulated in a liposome.

22. A composition including the recombinant polypeptide of embodiment any one of embodiments 1-9 or the recombinant polynucleotide of any one of embodiments 10-16 and a pharmaceutically acceptable carrier.

23. The composition of embodiment 22, wherein the recombinant polypeptide or the recombinant polynucleotide is encapsulated in a liposome.

24. A method including contacting skin of a subject with a therapeutically effective amount of a composition including the recombinant polypeptide of any one of embodiments 1-9 or the recombinant polynucleotide of any one of embodiments 10-16.

25. The method of embodiment 24, which is a method for: repairing UV-induced DNA damage in skin of a subject; reducing the number of tumors, size of tumors and/or total tumor burden in a subject exposed to UV irradiation; and/or treating or reducing the risk of a skin disorder in a subject.

26. The method of embodiment 25, wherein the skin disorder is melanoma, non-melanoma skin cancer (NMSC), actinic keratosis (AK), angiofibroma, pachyonychia congenita, or xeroderma pigmentosum.

27. The method of embodiment 26, wherein the NMSC includes basal cell carcinoma (BCC), squamous cell carcinoma (SCC), Merkel cell carcinoma, cutaneous (skin) lymphoma, Kaposi sarcoma, skin adnexal tumors and sarcomas.

28. The method of embodiment 27, wherein the subject has xeroderma pigmentosum or is an organ-transplant patient.

29. The method of embodiment 28, wherein the frequency of AK is reduced.

30. A method for treating or reducing UV-induced immunosuppression in a subject in need thereof including administering a therapeutically effective amount of the recombinant polypeptide of any one of embodiments 1-9 or the recombinant polynucleotide of any one of embodiments 10-16 to the subject to treat or reduce UV-induced immunosuppression in the subject as compared to a subject in need thereof not administered a therapeutically effective amount of the recombinant polypeptide of any one of embodiments 1-9 or the recombinant polynucleotide of any one of embodiments 10-16.

31. The method of embodiment 30, wherein an increase in repair of CPDs and/or 6-4 PPs occurs in the subject administered the therapeutically effective amount of the recombinant polypeptide or the recombinant polynucleotide as compared to the subject not administered the therapeutically effective amount of the recombinant polypeptide or the recombinant polynucleotide.

32. A method for decreasing the severity of a UV-induced inflammatory response in a subject in need thereof including administering a therapeutically effective amount of the recombinant polypeptide of any one of embodiments 1-9 or the recombinant polynucleotide of any one of embodiments 10-16 to the subject to decrease the severity of the UV-induced inflammatory response in the subject as compared to a subject in need thereof not administered a therapeutically effective amount of the recombinant polypeptide of any one of embodiments 1-9 or the recombinant polynucleotide of any one of embodiments 10-16.

33. The method of embodiment 32, wherein the amount of circulating lymphocytes, monocytes and/or eosinophils are reduced in the subject administered the therapeutically effective amount of the recombinant polypeptide or the recombinant polynucleotide.

### Second Set of Exemplary Embodiments

1. A recombinant polypeptide including a truncated UV damage endonuclease (UVDE) including at least one heterologous targeting sequence at the carboxy-terminus (C-terminus) of the UVDE, wherein the truncation is amino-terminal (N-terminal) to a conserved region of the UVDE required for enzymatic activity.

2. The recombinant polypeptide of embodiment 1, wherein the truncated UVDE is Schizosaccharomyces pombe Uve1p lacking the first 228 amino acids of SEQ ID NO: 16.

3. The recombinant polypeptide of embodiment 1 or 2, wherein the at least one heterologous targeting sequence includes a cell penetrating peptide.

4. The recombinant polypeptide of embodiment 3, wherein the cell penetrating peptide includes a transactivator of transcription (TAT) peptide from human immunodeficiency virus.

5. The recombinant polypeptide of embodiment 4, wherein TAT is an amino acid sequence set forth in SEQ ID NO: 2.

6. The recombinant polypeptide of embodiment 1 or 2, wherein the at least one heterologous targeting sequence includes a nuclear localization signal (NLS).

7. The recombinant polypeptide of embodiment 6, wherein the NLS is an amino acid sequence selected from SEQ ID NOs: 6, 10, 17, 18, and 20-61.

8. The recombinant polypeptide of any of embodiments 1-7, wherein the at least one heterologous targeting sequence includes a NLS and a TAT protein transduction domain.

9. The recombinant polypeptide of embodiment 8, wherein the NLS is an amino acid sequence selected from SEQ ID NOs: 6, 10, 17, 18, and 20-61 and the TAT is an amino acid sequence set forth in SEQ ID NO: 2.

10. The recombinant polypeptide of embodiment 8 or 9, wherein the NLS-TAT includes an amino acid sequence set forth in SEQ ID NO: 4.

11. The recombinant polypeptide of embodiment 1 or 2, wherein the recombinant polypeptide is encoded by an amino acid sequence selected from SEQ ID NOs: 12 and 14.

12. The recombinant polypeptide of any one of embodiments 1-10, wherein the recombinant polypeptide further includes one or more purification tags.

13. The recombinant polypeptide of embodiment 12, wherein the one or more purification tags includes a hexahistidine tag.

14. The recombinant polypeptide of any one of embodiments 1-13, wherein the recombinant polypeptide is encapsulated in a liposome.

15. The recombinant polypeptide of embodiment 14, wherein the liposome includes 1,2-dioleoyl-sn-glycero-3-phosphoethanolamine (DOPE), 1,2-dioleoyl-sn-glycero-3-phosphocholine (DOPC), cholesteryl hemisuccinate (CHEMS) and oleic acid in a 2:2:5:1 molar ratio.

16. The recombinant polypeptide of embodiment 14 or 15, wherein the liposome is in a 0.75% (w/v) hydrogel solution.

17. The recombinant polypeptide of any one of embodiments 14-16, wherein the recombinant polypeptide is at a concentration of 1 μg/mL to 50 μg/mL.

18. The recombinant polypeptide of any one of embodiments 14-17, wherein the recombinant polypeptide is at a concentration of 10 μg/mL.

19. A recombinant polynucleotide encoding a truncated UV damage endonuclease (UVDE) sequence including at least one heterologous targeting sequence at the 3′ end of the UVDE sequence, wherein the truncation is 5′ to a conserved region of the UVDE sequence required for enzymatic activity.

20. The recombinant polynucleotide of embodiment 19, wherein the truncated UVDE sequence is Schizosaccharomyces pombe Uve1 lacking the first 684 nucleotides of SEQ ID NO: 15.

21. The recombinant polynucleotide of embodiment 19 or 20, wherein the at least one heterologous targeting sequence includes a cell penetrating peptide sequence.

22. The recombinant polynucleotide of embodiment 21, wherein the cell penetrating peptide sequence includes a transactivator of transcription (TAT) sequence from human immunodeficiency virus.

23. The recombinant polynucleotide of embodiment 22, wherein the TAT sequence has a nucleotide sequence set forth in SEQ ID NO: 1.

24. The recombinant polynucleotide of any one of embodiments 19-23, wherein the at least one heterologous targeting sequence includes a nuclear localization signal (NLS) sequence.

25. The recombinant polynucleotide of embodiment 24, wherein the NLS has a nucleotide sequence selected from SEQ ID NOs: 5 and 9.

26. The recombinant polynucleotide of embodiment 19 or 20, wherein the at least one heterologous targeting sequence includes a NLS sequence and a TAT cell penetrating peptide sequence.

27. The recombinant polynucleotide of embodiment 26, wherein the NLS sequence has a nucleotide sequence selected from SEQ ID NOs: 5 and 9 and the TAT sequence has a nucleotide sequence set forth in SEQ ID NO: 1.

28. The recombinant polynucleotide of embodiment 27, wherein the NLS-TAT has a nucleotide sequence set forth in SEQ ID NO: 3.

29. The recombinant polynucleotide of embodiment 19 or 20, wherein the recombinant polynucleotide has a nucleotide sequence selected from SEQ ID NOs: 11 and 13.

30. The recombinant polynucleotide of any one of embodiments 19-28, wherein the recombinant polynucleotide further includes one or more purification tag sequences.

31. The recombinant polynucleotide of embodiment 30, wherein the one or more purification tag sequences includes a hexahistidine tag sequence.

32. A vector including the polynucleotide of any one of embodiments 19-31.

33. A host cell including the recombinant polypeptides of any one of embodiments 1-18 or the vector of embodiment 32.

34. A topical formulation including a therapeutically effective amount of the recombinant polypeptide of any one of embodiments 1-18 or the recombinant nucleotide of any one of embodiments 19-31.

35. The topical formulation of embodiment 34, wherein the topical formulation is incorporated in a lotion, a cream, a paste, a powder, a sunscreen, a liquid, an aerosol, a suspension, an emulsion, a hydrogel, a plaster, a patch, a bandage, or a film.

36. The topical formulation of embodiment 34 or 35, wherein the recombinant polypeptide or the recombinant polynucleotide is encapsulated in a liposome.

37. The topical formulation of embodiment 36, wherein the liposome includes 1,2-dioleoyl-sn-glycero-3-phosphoethanolamine (DOPE), 1,2-dioleoyl-sn-glycero-3-phosphocholine (DOPC), cholesteryl hemisuccinate (CHEMS) and oleic acid in a 2:2:5:1 molar ratio.

38. The topical formulation of embodiment 36 or 37, wherein the liposome is in a 0.75% (w/v) hydrogel solution.

39. The topical formulation of any one of embodiments 36-38, wherein the recombinant polypeptide is at a concentration of 1 μg/mL to 50 μg/mL.

40. The topical formulation of any one of embodiments 36-39, wherein the recombinant polypeptide is at a concentration of 10 μg/mL.

41. A composition including the recombinant polypeptide of any one of embodiments 1-18 or the recombinant polynucleotide of any one of embodiments 19-31 and a pharmaceutically acceptable carrier.

42. The composition of embodiment 41, wherein the recombinant polypeptide or the recombinant polynucleotide is encapsulated in a liposome.

43. The composition of embodiment 42, wherein the liposome includes 1,2-dioleoyl-sn-glycero-3-phosphoethanolamine (DOPE), 1,2-dioleoyl-sn-glycero-3-phosphocholine (DOPC), cholesteryl hemisuccinate (CHEMS) and oleic acid in a 2:2:5:1 molar ratio.

44. The composition of embodiment 42 or 43, wherein the liposome is in a 0.75% (w/v) hydrogel solution.

45. The composition of any one of embodiments 41-44, wherein the recombinant polypeptide is at a concentration of 1 μg/mL to 50 μg/mL.

46. The composition of any one of embodiments 41-45, wherein the recombinant polypeptide is at a concentration of 10 μg/mL.

47. A method for repairing UV-induced DNA damage in skin of a subject including contacting the skin of the subject with a therapeutically effective amount of a composition including the recombinant polypeptide of any one of embodiments 1-18 or the recombinant polynucleotide of any one of embodiments 19-31.

48. A method for reducing the number of tumors, size of tumors and/or total tumor burden in a subject exposed to UV irradiation, including administering a therapeutically effective amount of the recombinant polypeptide of any one of embodiments 1-18 or the recombinant polynucleotide of any one of embodiments 19-31 to the subject to reduce the number of tumors, size of tumors and/or total tumor burden as compared to a subject exposed to UV irradiation not administered a therapeutically effective amount of the recombinant polypeptide of any one of embodiments 1-18 or the recombinant polynucleotide of any one of embodiments 19-31.

49. A method for treating or reducing the risk of a skin disorder in a subject including administering a therapeutically effective amount of the recombinant polypeptide of any one of embodiments 1-18 or the recombinant polynucleotide of any one of embodiments 19-31 to the subject.

50. The method of embodiment 49, wherein the skin disorder is melanoma, non-melanoma skin cancer (NMSC), actinic keratosis (AK), angiofibroma, pachyonychia congenita, or xeroderma pigmentosum.

51. The method of embodiment 50, wherein the NMSC includes basal cell carcinoma (BCC), squamous cell carcinoma (SCC), Merkel cell carcinoma, cutaneous (skin) lymphoma, Kaposi sarcoma, skin adnexal tumors and sarcomas.

52. The method of any one of embodiments 49-51, wherein the subject has xeroderma pigmentosum or is an organ-transplant patient.

53. The method of embodiment 50, wherein the frequency of AK is reduced.

54. A method for treating or reducing UV-induced immunosuppression in a subject in need thereof including administering a therapeutically effective amount of the recombinant polypeptide of any one of embodiments 1-18 or the recombinant polynucleotide of any one of embodiments 19-31 to the subject to treat or reduce UV-induced immunosuppression in the subject as compared to a subject in need thereof not administered a therapeutically effective amount of the recombinant polypeptide of any one of embodiments 1-18 or the recombinant polynucleotide of any one of embodiments 19-31.

55. The method of embodiment 54, wherein an increase in repair of CPDs and/or 6-4 PPs occurs in the subject administered the therapeutically effective amount of the recombinant polypeptide of any one of embodiments 1-18 or the recombinant polynucleotide of any one of embodiments 19-31 as compared to the subject not administered the therapeutically effective amount of the recombinant polypeptide of any one of embodiments 1-18 or the recombinant polynucleotide of any one of embodiments 19-31.

56. A method for decreasing the severity of a UV-induced inflammatory response in a subject in need thereof including administering a therapeutically effective amount of the recombinant polypeptide of any one of embodiments 1-18 or the recombinant polynucleotide of any one of embodiments 19-31 to the subject to decrease the severity of the UV-induced inflammatory response in the subject as compared to a subject in need thereof not administered a therapeutically effective amount of the recombinant polypeptide of any one of embodiments 1-18 or the recombinant polynucleotide of any one of embodiments 19-31.

57. The method of embodiment 56, wherein the amount of circulating lymphocytes, monocytes and/or eosinophils are reduced in the subject administered the therapeutically effective amount of the recombinant polypeptide of any one of embodiments 1-18 or the recombinant polynucleotide of any one of embodiments 19-31.

Example 1. The molecular basis for ultraviolet (UV) light-induced nonmelanoma and melanoma skin cancers centers on cumulative genomic instability caused by inefficient DNA repair of dipyrimidine photoproducts. Inefficient DNA repair and subsequent translesion replication past these DNA lesions generate distinct molecular signatures of tandem CC to TT and C to T transitions at dipyrimidine sites. Since previous efforts to develop experimental strategies to enhance the repair capacity of basal keratinocytes have been limited, the inventors of the present application have engineered the N-terminally truncated form (Δ228) of UV endonuclease (UVDE) from Schizosaccharomyces pombe to include a TAT cell-penetrating peptide sequence with or without a nuclear localization signal (NLS) and with or without a HIS6 purification tag: UVDE-TAT±His6 and UVDE-NLS-TAT±His6. Further, a NLS was engineered onto a pyrimidine dimer glycosylase from Paramecium bursaria chlorella virus-1 (cv-pdg-NLS-His6). Purified enzymes were encapsulated into liposomes and topically delivered to the dorsal surface of SKH1 hairless mice in a UVB-induced carcinogenesis study. Total tumor burden was significantly reduced in mice receiving either UVDE-TAT-His6 or UVDE-NLS-TAT-His6 versus control empty liposomes and time to death was significantly reduced with the UVDE-NLS-TAT-His6. These data suggest that efficient delivery of exogenous enzymes for the initiation of repair of UVB-induced DNA damage may protect from UVB induction of squamous and basal cell carcinomas. At least some of the subject matter descried in this example was published as Sha et al., Scientific Reports 8:705, 2018; DOI:10.1038/s41598-017-17940-8.

### Methods

Cloning, Expression, Purification, and Encapsulation of UVDE Constructs. Engineering UVDE-TAT-His6 and UVDE-NLS-TAT-His6. Bacterial expression plasmids for UVDE-TAT-His6 and UVDE-NLS-TAT-His6 were created in a pET21b expression vector backbone to include a C-terminal hexahistidine tag. The UVDE construct was generated by subcloning a truncation of the sequence for the S. pombe uve1 gene (GenBank accession number NP 596165.1) between the NdeI and HindIII sites into the multiple cloning sequence, leaving a short linker encoding for LAAALE (SEQ ID NO: 7) between the last amino acid for the protein (K) and the hexahistidine tag (HHHHHH, SEQ ID NO: 8; the hexahistidine tag is encoded by CACCACCACCACCACCAC, SEQ ID NO: 62). The truncated UVDE lacking amino acid residues 1 through 228 is encoded by nucleotide sequence SEQ ID NO: 63 shown in FIG. 15 and amino acid sequence SEQ ID NO: 64 shown in FIG. 16. The sequence encoding the AAALE (SEQ ID NO: 65) was removed and substituted with sequences to encode the TAT peptide (YGRKKRRQRRR, SEQ ID NO: 2) with the intervening DNA sequence being 5′-TATGGCCGCAAAAAGCGCCGTCAGCGCCGTCGC-3′ (SEQ ID NO: 1) to generate the UVDE-TAT expression construct. For the UVDE-NLS-TAT-His6 expression construct, the NLS nucleotide sequence used is 5′-CCAAAGAAGAGGAAAAGGAGG-3′ (SEQ ID NO: 5) encoding PKKRKRR (SEQ ID NO: 6). Site-directed mutagenesis was used to insert the NLS-TAT sequence (PKKRKRRLYGRKKRRQRRR, SEQ ID NO: 4), encoded by 5′-CCAAAGAAGAGGAAAAGGAGGCTATATGGCCGCAAAAAGCGCCGTCAGCGCCGTCGC-3′ (SEQ ID NO: 3), yielding the UVDE-NLS-TAT expression construct.

Fermentation and purification of UVDE-TAT-His6 and UVDE-NLS-TAT-His6. Single colony isolates of BL21(DE3) with pET-UVDE-TAT-His6 or UVDE-NLS-TAT-His6 were grown overnight in 125 mL of Terrific Broth (TB) (Corning 46-055-CM, 12.0 g casein peptone, 4.0 ml glycerol, 2.31 g KH₂PO₄, 12.54 g K₂HPO₄, 24.0 g yeastolate) with 100 μg/L carbenicillin. A total of 10 mL of the overnight culture was used to inoculate a 1 L TB with 100 μg/L carbenicillin. The culture was shaken at 250 rpm at 37° C. until an OD₆₀₀ of 4 was achieved. Protein expression was induced overnight with 0.2 mM IPTG at 16° C. Cell paste was harvested by centrifugation and frozen at −80° C. before purification.

Cell paste was resuspended in lysis buffer (20 mM HEPES, pH 7.5, 20 mM imidazole, pH 7.5, 500 mM NaCl, 1 mM MnCl₂, 10% glycerol, 0.5 mM PMSF, 14 mM β-mercaptoethanol) and lysed using a microfluidizer (Microfluidics, Inc., Model 110L). The resulting lysate was cleared by centrifugation at 38,400 g for 60 min at 4° C. The cleared lysate was applied to a 5 mL HisTrap HP column (GE Healthcare). The column was washed extensively with lysis buffer before the protein was eluted with a gradient of 20-300 mM imidazole and followed with 400 mM imidazole. Fractions containing UVDE-TAT-His6 or UVDE-NLS-TAT-His6 were pooled and loaded to a 5 mL HiTrap Heparin HP column (GE Healthcare). After extensive washes with Heparin Buffer A (25 mM HEPES pH 7.5, 500 mM NaCl, 10% glycerol, 1 mM MnCl₂, 0.5 mM PMSF, 14 mM β-mercaptoethanol), the protein was eluted with a gradient of 500-1,000 mM NaCl. The heparin pool was concentrated and dialyzed into storage buffer (25 mM HEPES, pH 7.5, 500 mM NaCl, 1 mM MnCl₂, 10% glycerol, 1 mM TCEP) or further purified by HiPrep 26/60 Sephacryl S-200 HR size exclusion column (GE Healthcare) in the storage buffer. The protein was concentrated using Amicon Ultra-15 centrifugal filter units (EMD Millipore), flash frozen in liquid nitrogen, and stored at −80° C. The final yields of UVDE-TAT-His6 and UVDE-NLS-TAT-His6 were 106 and 14 mg/L of E. coli culture, respectively. Photographs of the Coomassie-stained gels represent a single stained gel.

Engineering cv-pdg-NLS-His6. In order to create a cv-pdg-NLS-His6 expression vector (pcv-pdgNLS-His6), the coding sequence for the gene encoding cv-pdg (from the Paramecium bursaria Chlorella virus 1 genome, PBCV-1: GenBank accession number JF411744.1; U.S. Pat. No. 6,723,548) was engineered to contain a six-nucleotide spacer and a sequence encoding a nuclear localization signal (NLS) between the final codon and the stop translation codon (TGA). The NLS sequence (5′-CCCGGGCCAAAGAAAAAGAGGAAGAGGCTA-3″, SEQ ID NO: 9) encodes for the amino acid sequence PGPKKKRGRL (SEQ ID NO: 10). The DNA sequence encoding the NLS was cloned into pET24a between the Nde1 and HindIII restriction sites. The sequences of all gene constructs were verified prior to expression studies. The plasmid was transformed into BL21 (DE3) cells and glycerol stocks were made for expression and purification.

Large-scale fermentation and purification of cv-pdg-NLS-His6. Fed-batch culture was carried out in a stirred tank fermenter, with a base medium of minimal R/2 medium (2 g of (NH₄)₂HPO₄, 6.75 g of KH₂PO₄, 0.85 g of citric acid, and 0.7 g of MgSO₄·7H₂O per liter) supplemented with 20 g/L yeast extract and 0.5 ml/L trace metal solution (27 g of FeCl₃·6H₂O, 2 g of ZnCl₂·4H₂O, 1 g of CuCl₂, 2 g of CoCl₂·6H₂O, 0.5 g of H₃BO₃, 1 g of CaCl₂·2H₂O, and 2 g of Na₂MoO₄·6H₂O per liter of 1.2 N HCl), 8 g/L glucose, 10 g/L glycerol, 25 mg/L kanamycin, and 0.1 ml/L antifoam. An overnight seed culture was prepared from a glycerol stock of BL21(DE3) with pET24a-cv-pdg-NLS in the supplemented R/2 medium without glycerol and antifoam. The batch phase was run at 37° C. The pH was controlled at 7.0 by additions of 4 M NH₄OH or 4 M H₃PO₄. The dissolved-oxygen concentration was controlled at 30% of air saturation (dO₂ controller was set to cascade between agitation speed and O₂ supplementation). When the OD₆₀₀ reached 10, a glucose feed (50% glucose, 7 g/L MgSO₄) was initiated to maintain glucose level above 2 g/L. When the OD₆₀₀ reached 42, the feed was switched to glycerol (50% glycerol, 7 g/L MgSO₄) and the temperature was lowered to 19° C. over the course of 30 min. Protein expression was induced by adding galactose to 2 g/L in one bolus. Biomass was harvested 5 hr post induction.

Cell paste was resuspended in lysis buffer (25 mM Tris-HCl, pH8.0, 150 mM NaCl) and processed using a microfluidizer (Model M110L, Microfluidics, Inc.). The resulting lysate was cleared by centrifugation at 38,400 g for 60 min at 4° C. The cleared lysate was applied to a Q Sepharose FF column (GE Healthcare). The Q flow-through was collected and loaded to a SP Sepharose HP column (GE Healthcare). The column was washed extensively with IEX Buffer A (25 mM Tris-HCl, pH 8.0, 150 mM NaCl) before step-elution with 0.2 M, 0.3 M, 0.4 M and 0.5 M NaCl in IEX Buffer B (25 mM Tris-HCl, pH 8.0, 0.5 M NaCl). Fractions containing cv-pdg-NLS from 0.4 M NaCl elution were pooled and further purified by size exclusion using a HiPrep 26/60 Sephacryl S-100 HR column (GE Healthcare) in 1× PBS, pH 7.4. The protein was concentrated by ultrafiltration, flash frozen, and stored at −80° C.

Encapsulation of Repair Enzymes. 1,2-dioleoyl-sn-glycero-3-phosphocholine (DOPC), 1,2-dioleoyl-sn-glycero-3-phosphoethanolamine (DOPE), and cholesteryl hemisuccinate (CHEMS) were purchased from Avanti Polar lipids (Alabaster, Ala.). Oleic acid was purchased from Sigma Aldrich (St Louis, Mo.). Carbomer 940 was purchased from Makingcosmetics Inc. (Snoqualmie, Wash.). Track-etch polycarbonate membranes, engineered with pores 2 μm, 1 μm, 400 nm and 200 nm were purchased from Millipore (Billerica, Mass.), Lipex liposome extruder was from Northern Lipids (Burnaby, Canada). Dermal syringes and other formulation packaging materials were purchased from Medi-Dose Inc (Warminster, Pa.). All common lab chemicals and reagents were from Sigma-Aldrich and Thermo Fisher Scientific (Waltham, Mass.).

Liposomes were composed of 2:2:5:1 molar ratio of DOPE:DOPC:CHEMS:Oleic Acid encapsulating cv-pdg-NLS-His6, UVDE-TAT-His6, and UVDE-NLS-TAT-His6 at 10 μg/mL each. Lipids were dissolved in chloroform and the solvent was evaporated using a Büchi (RE-121) rotary evaporator (Flawil, Switzerland) under vacuum for 4 hr. A thin lipid film was formed. The lipid film was hydrated with the repair enzymes in PBS buffer for 2 hr at 37° C. The milky solution of liposomes was extruded consecutively 20 times through 2 μm, 1 μm, 400 nm and 200 nm polycarbonate membrane filter using a Lipex extruder connected to high pressure argon cylinder. The sizes of the liposomes were measured using Malvern Zetasizer nano ZS90 (Malvern, United Kingdom).

A total of 10 g of Carbomer 940 was added to 1000 mL of PBS to form a 1% hydrogel solution. The solution was mixed using a Gowe® Electrical Compact Laboratory mixer. The pH of the mixture was adjusted to 7.4 by slow addition of NaOH. At pH above 6 the mixture forms a gel structure. The hydrogel was mixed thoroughly for 3 hr at room temperature. Liposomes were added to 1% hydrogel solution to make the final hydrogel concentration of 0.75%. The solutions were mixed thoroughly for 1 hr at room temperature and packaged in dermal syringes. The liposomal formulations contained 1 mM MnCl₂ in all stages of purification (Sha et al., Scientific Reports 8:705, 2018).

Animal Carcinogenesis and Tissue Preparation. Female SKH1 hairless mice (6 weeks old) were obtained from Jackson Laboratories and group-housed at 5 mice per box.

A total of 50 SKH1 hairless mice were randomly divided into 4 treatment groups (10 mice each), while an additional 10 mice were held with no treatments for observation of spontaneous tumor formation. After mice were housed for 2 weeks, all mice in each treatment group: 1. control empty liposome, 2. liposomes containing UVDE-TAT-His6, 3. liposomes containing UVDE-NLS-TAT-His6 or 4. liposomes containing cv-pdg-NLS-His6 were treated as follows. One hr prior to UVB irradiation, 0.2 mL of the liposomal formulations were uniformly applied to the dorsal surface of each mouse using a pre-moistened cotton swab. This volume of liposome was absorbed within 3-5 min following application. Mice were irradiated 1 hr after application of the liposome in the morning on Monday, Wednesday, and Friday with UVB in a ventilated 8-chambered plexiglass box that was covered with a UVB-transmissible quartz plate. During the first 3 weeks with 9 total exposures, all irradiated mice were exposed to 225 J/m² per exposure to allow photoaging without sunburn or blistering. Following this 3-week period of UVB acclimation, mice received 22 kJ/m²/week and all mice were examined at least three times per week for an additional 30 weeks. Although mice were monitored at least 3 times per week for tumor formation, the recording of tumor sizes was performed at 15, 18, 19, 20, 21, 22, 23, 24, 26, 28, 30, 32, and 33 weeks. At 23 weeks of UVB exposures, all mice were photographed for a visual record of the condition of the dorsal skin. Mice that developed either tumors >8 mm in diameter or ulcerated tumors were euthanized. Immediately following euthanasia, epidermal and dermal tissues were collected from representative tumor and non-tumor sections of the backs of the mice. Additionally, at the termination of the study (after 33 weeks), mice from the untreated group (no liposomes and no UVB treatment) were also euthanized and representative skin samples harvested. Tissues were fixed in aqueous-buffered zinc formalin (4% (w/v) formaldehyde and 600 ppm zinc) and after 2 days transferred into 70% (v/v) ethanol. Tissues were paraffin embedded and sections cut for hematoxylin and eosin (H&E) staining. Photographs were taken on a Zeiss ApoTome 2, using the Zeiss AxioCam 506 CCD color camera with no internal magnification. Scale bars were added using Image J software.

Biostatistical Methods. The two time-to-event outcomes (death and first 2 mm tumor) were depicted graphically with nonparametric Kaplan-Meier curves for each treatment group and compared using the log-rank test. Reported log-rank p-values for pairwise comparisons between treatment groups were adjusted for multiplicity using the Tukey method (Kramer, Biometrics 12, 307-310, 1956). Cox regression was applied to the time-to-death outcome to estimate hazard ratios for the risk of tumor progression-induced euthanasia when comparing each active treatment group to the control group. The Cox model assumption that the risks of death are proportional over the study period for the groups being compared was checked and found to be justifiable.

There were no missing values for total tumor size comparisons at either of the two time points of interest (23 weeks and 33 weeks) since a mouse's last measured value was carried over if it had been euthanized prior to the time point. Although the sample distributions of total tumor size were often positively skewed, this outcome was nonetheless analyzed using a one-way ANOVA as this method is known to be robust to deviations from the normality assumption. Moreover, discrepancies in the variance of total tumor size between treatment groups discouraged the use of a nonparametric method that assumes the distributions have equal spread. At each of the two time points of interest, the significance of total tumor burden differences between treatment groups was quantified with Tukey-adjusted pairwise p-values from the corresponding ANOVA model.

Statistical significance was ascribed to all effects with multiplicity-adjusted p-values <0.05. Statistical analysis was done in SAS 9.4 and R 4.0.

### Results.

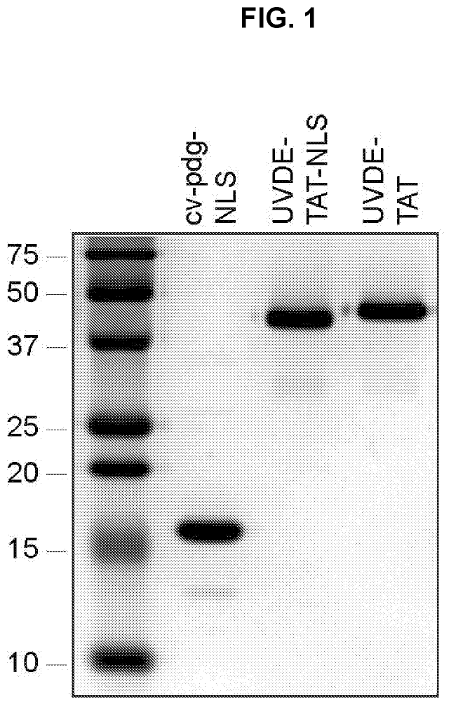

Preparation of Dipyrimidine DNA Repair Enzymes for Topical Delivery. Since mammalian cells exclusively use NER to initiate the repair of dipyrimidine photoproducts, the focus of this study was to determine if the induction of UVB-induced NMSCs could be significantly reduced relative to controls through the delivery of an enzyme that repairs both CPDs and 6-4 PPs. To facilitate and maximize cellular delivery of UVDE, a sequence encoding a protein transduction peptide, TAT (YGRKKRRQRRR, SEQ ID NO: 2), was engineered onto the C-terminus of UVDE (UVDE-TAT-His6). Further, since the catalytically-active, truncated form of UVDE lacks its natural NLS site, the E. coli expression vector was also modified to insert the 7-amino acid NLS (PKKRKRR, SEQ ID NO: 6) at the C-terminus (UVDE-NLS-TAT-His6). The sequences of the complete genes were confirmed prior to expression studies. Protein expression and purification were optimized, with a final yield of UVDE-TAT-His6 and UVDE-NLS-TAT-His6 of 106 mg/L and 14 mg/L, respectively. Further, the expression construct for cv-pdg was engineered to contain a 10 amino acid NLS (PGPKKKRGRL, SEQ ID NO: 10) on the C-terminal portion of the enzyme and purified, with the final yield being 55 mg/mL. The purities of these enzymes are shown in FIG. 1. (It is noted that the Figures do not explicitly refer to the His6 purification tag, though that tag is present as indicated in the description and Figure Legends.)

To topically deliver these enzymes to mouse skin, each protein was encapsulated at 10 μg/mL into liposomes using a formulation previously described for the T4-pdg studies (Ceccoli et al., J Invest Dermatol 93, 190-194, 1989). Details for liposomal preparation are given in the Methods section. All liposomes were formulated with hydrogel to yield a final concentration of 0.75%, which allowed uniform distribution and absorbance into the mouse skin.

UVB-induced Carcinogenesis. A total of 40 SKH1 hairless mice were assigned to 4 treatment groups in the following experimental design: 1) empty liposome control, 2) UVDE-TAT-His6, 3) UVDE-NLS-TAT-His6, and 4) cv-pdg-NLS-His6. An additional 10 mice were not treated with liposomes or UVB to assess frequencies of spontaneous skin lesions; however, these mice never developed any spontaneous skin tumors and are not included in further analyses. Beginning at 8 weeks of age, 0.2 mL of each liposomal formulation was uniformly applied to the dorsal skin of each mouse using a pre-moistened cotton swab. This amount was absorbed to apparent dryness within 3-5 min after application. After 1 hr, mice were exposed to increasing doses of UVB irradiation in an 8-compartment chamber that was covered with a quartz plate to allow full UVB penetrance. This chamber maximized exposures to the backs of the mice.

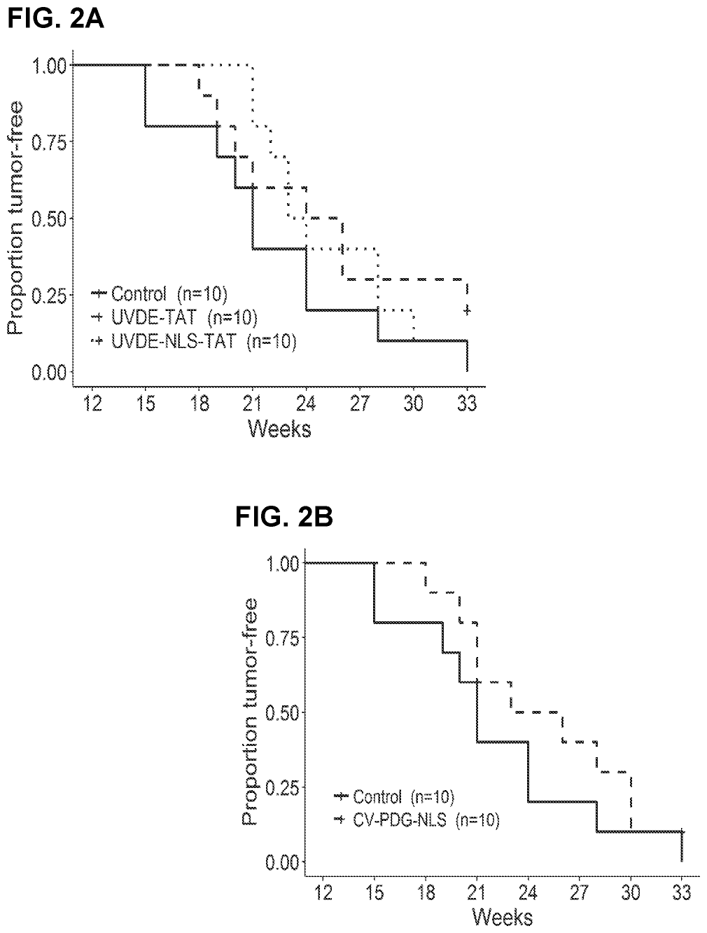

To prevent significant skin irritation and burning, a UVB dose escalation strategy was used for the initial irradiations. As expected, following 9 UVB exposures (a total exposure of 2.0 kJ/m²) over the course of 3 weeks, the dorsal skin showed evidence of mild redness, slight loss in elasticity, and thickening, without any evidence of sunburn. All mice continued to receive average weekly doses of 22 kJ/m² and all mice were monitored three times per week for skin lesions. The earliest time for the formation of confirmed squamous cell carcinomas in the control empty liposome group was at 15 weeks, with a total cumulative exposure of 217 kJ/m². Analyses ofthe length of time to form the first tumor (2 mm diameter) in each irradiated mouse revealed that although there were trends for a delay in time to first tumor in all 3 active enzyme groups (shown in FIGS. 2A and 2B and quantified by the median in Table 3), these delays were not statistically distinguishable from the control group, with nonsignificant p-values of 0.404 for UVDE-TAT-His6, 0.788 for UVDE-NLS-TAT-His6, and 0.668 for cv-pdg-NLS-His6.

TABLE 3
Time (weeks) to First 2 mm Tumor, Summary Statistics

| | | | Range of | Median time |
|---|---|---|---|---|
| | | # mice | times to | to first 2 |
| Treatment | Group Size | with a 2 | first 2 mm | mm tumor |
| group | (# mice) | mm tumor | tumor | (95% Cl) |
| Control | 10 | 10 | 15 to 33 weeks | 21 weeks |
| | | | | (15.0 to 24.0) |
| UVDE-TAT- | 10 | 8 | 18 to 33 weeks | 25 weeks |
| His6 | | | | (18.0 to 33.0) |
| UVDE-NLS- | 10 | 10 | 21 to 33 weeks | 23.5 weeks |
| TAT-His6 | | | | (21.0 to 28.0) |
| cv-pdg-NLS- | 10 | 9 | 18 to 33 weeks | 24.5 weeks |
| His6 | | | | (18.0 to 30.0) |

Note:
the sample median in each group is the same as the Kaplan-Meier estimated median because all mice were evaluated until euthanasia was required or the end of the study was reached (after 33 weeks of UVB).

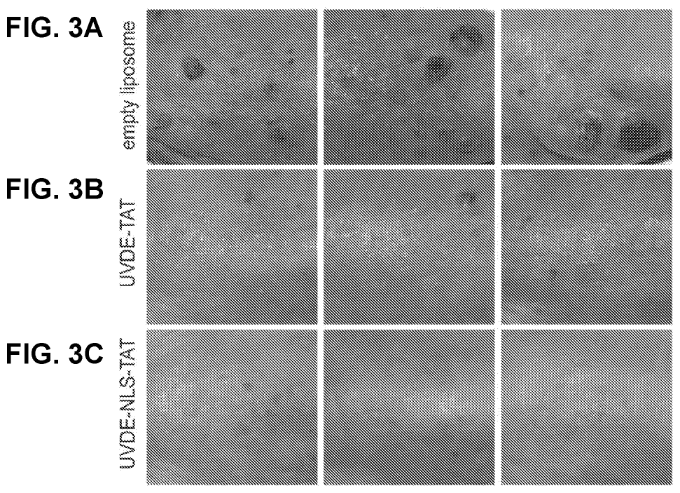

Following 23 weeks of UVB exposures, totaling 311 kJ/m², all mice were photographed for comparative analyses. Representative images of mice treated with control empty liposome, UVDE-TAT-His6, and UVDE-NLS-TAT-His6 are shown in FIGS. 3A-3C. Visual evaluation of the mice treated with control empty liposomes versus any of the enzyme-containing liposomes revealed a greater involvement of the dorsal surface area with various tumors, with 4 of the 10 mice having larger individual tumors that were >3 mm each. In contrast, mice treated with liposomes containing an enzyme showed less severe damage, with no tumors >3 mm observed in either form of UVDE, with only one tumor >3 mm in the cv-pdg-NLS-His6 group. These observations indicated a suppressive effect on tumor formation with the treatment of either of the repair enzymes.

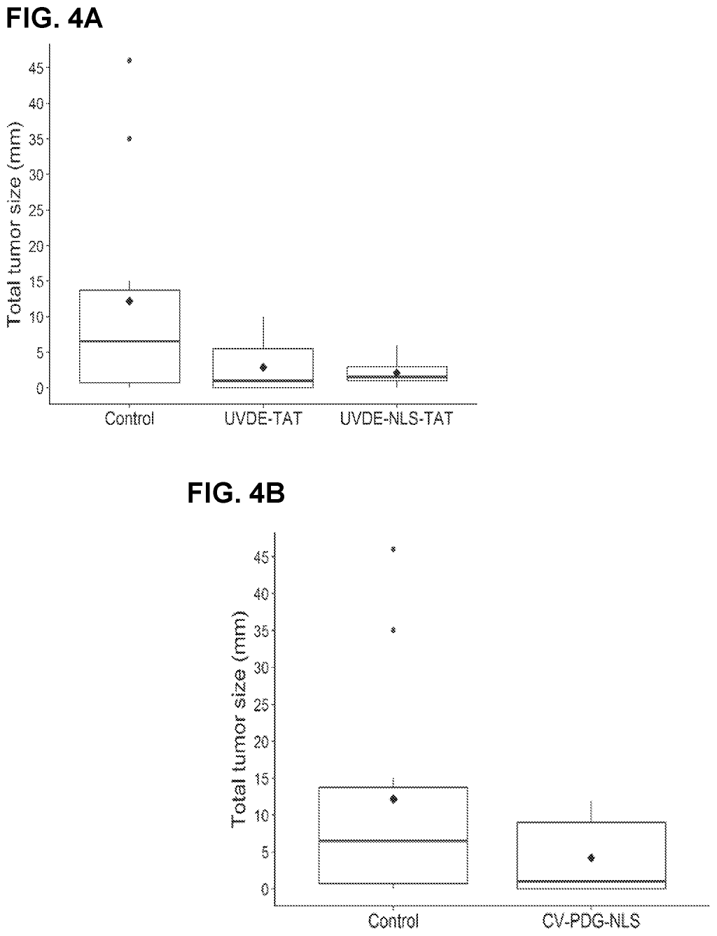

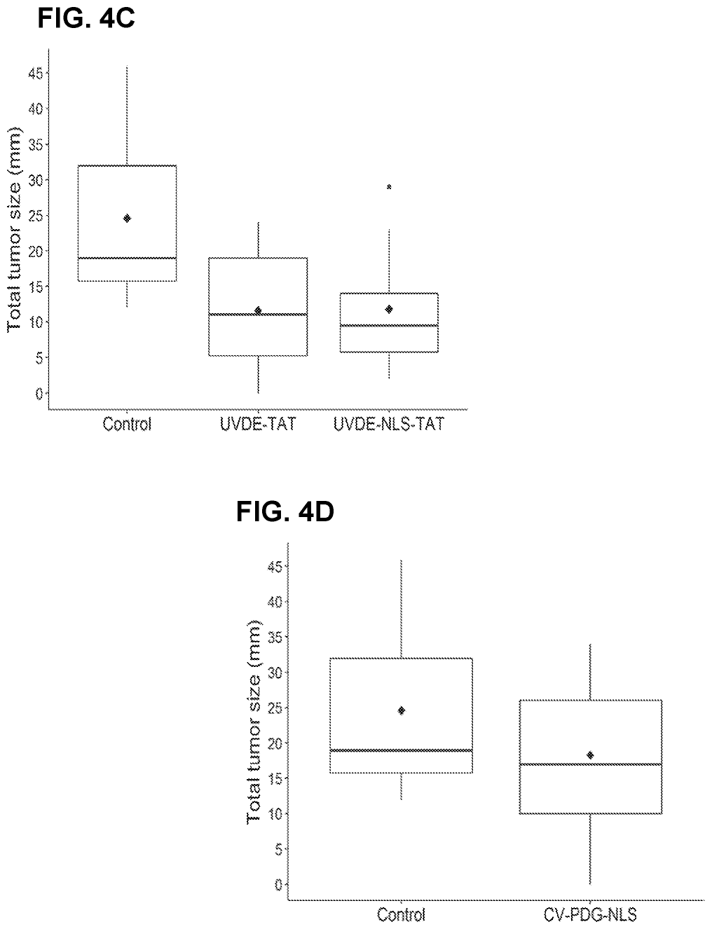

The total tumor burden (cumulative size of all tumors) for each mouse was analyzed at various time points, with the data for week 23 summarized in group-specific box plots for UVDE-TAT-His6 and UVDE-NLS-TAT-His6 (FIG. 4A) and for cv-pdg-NLS-His6 (FIG. 4B). At 23 weeks, the mean total tumor size per mouse for the empty, UVDE-TAT-His6, UVDE-NLS-TAT-His6, and cv-pdg-NLS-His6 liposome-treated groups were 12.2 mm, 2.9 mm, 2.1 mm, and 4.2 mm, respectively. The mean differences in total tumor size for UVDE-TAT-His6, UVDE-NLS-TAT-His6, and cv-pdg-NLS-His6 relative to the control group were not statistically significant (p=0.092, 0.058, and 0.179, respectively). The empty-liposome treated group had a total of 28 tumors with an aggregate total tumor size of 122 mm at 23 weeks. In contrast, the UVDE-TAT-His6, UVDE-NLS-TAT-His6, and cv-pdg-NLS-His6 groups had 18, 15, and 19 tumors, respectively, and aggregated tumor sizes of 29 mm, 21 mm, and 42 mm, respectively. The 23-week time point was chosen for analyses because only 1 mouse (in the control empty liposome treated group) had developed a tumor that required the mouse to be euthanized. In this and subsequent analyses of total tumor burden, mice that were euthanized prior to a specified time point were assigned tumor size values equal to their last measurements while alive. Imputation of these final measurements at post-death time points was important since exclusion of euthanized mice would bias the total tumor size comparisons, since mice with the largest tumor burdens (>8 mm for a single tumor) would be unavailable for the between-group analyses.

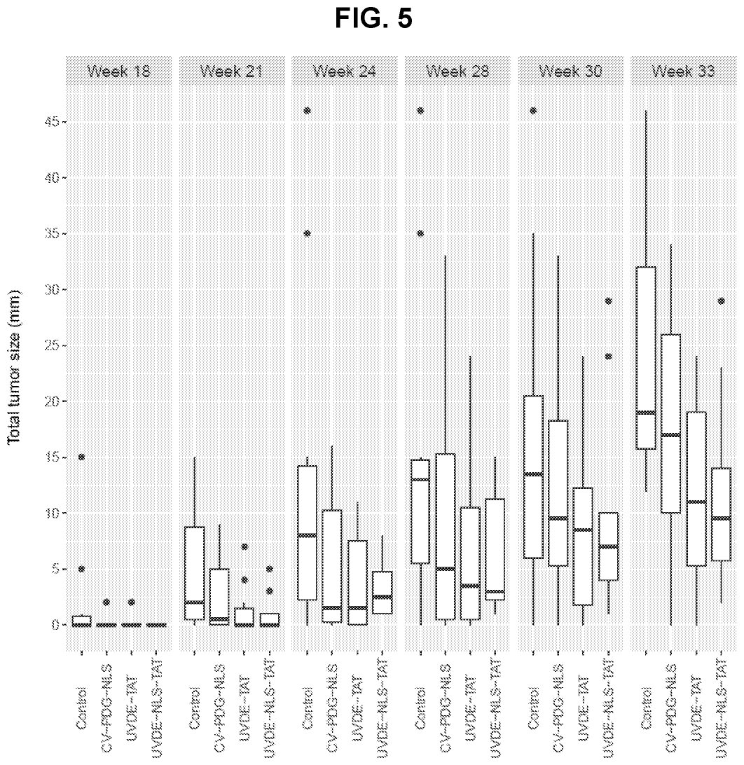

Comparable analyses were also made at 33 weeks with these data summarized in FIGS. 4C-4D. The mean total tumor size per mouse was 25 mm in the control group, 12 mm in both the UVDE treatment groups, and 18.3 mm in the cv-pdg-NLS-His6 group. These tumor burden averages, when compared to empty liposome controls, were statistically significant for UVDE-TAT-His6 (p=0.041) and UVDE-NLS-TAT-His6 (p=0.045), but not for cv-pdg-NLS-His6 (p=0.541). These findings demonstrate that, relative to the control empty liposomal treatment, application of liposomes containing UVDE-TAT-His6 or UVDE-NLS-TAT-His6 reduces the UVB-induced total tumor burden. FIG. 5 shows total tumor size by treatment group analyzed at 18 weeks, 21 weeks, 24 weeks, 28 weeks, 30 weeks and 33 weeks after UVB irradiation, summarized in group-specific box plots for empty liposome control, cv-pdg-NLS-His6, UVDE-TAT-His6 and UVDE-NLS-TAT-His6.

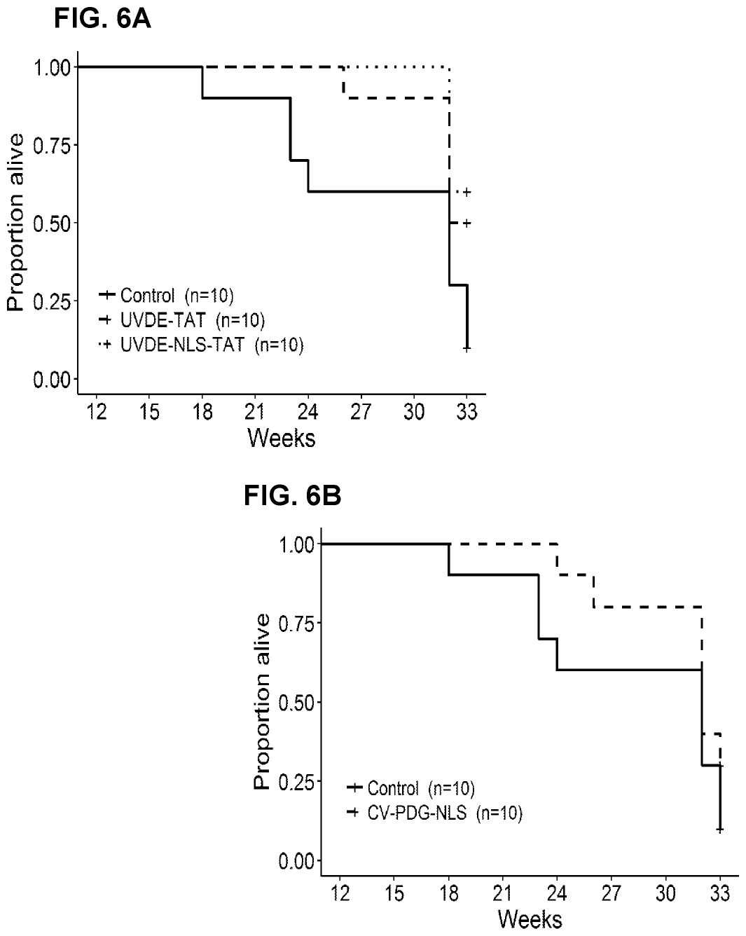

In addition, analyses were performed concerning time-to-death for each of the groups. To compare the risk of tumor-induced euthanasia for the three active enzyme groups relative to the control group, hazard ratios (HR) were estimated from a Cox regression model. Assuming proportional risks of euthanasia across all time points, estimated hazard (risk) ratios involving the control group were 0.35 (95% CI: 0.11, 1.05) for UVDE-TAT-His6, 0.25 (95% CI: 0.07, 0.81) for UVDE-NLS-TAT-His6, and 0.56 (95% CI: 0.21, 1.55) for cv-pdg-NLS-His6. Thus, UVDE-NLS-TAT-His6 mice had 75% less (95% CI: 93% to 19% less) risk of being euthanized than control mice during the treatment period. Without having to assume a constant risk ratio across time, analyses of Kaplan-Meier survival curves of control vs UVDE-TAT-His6 and UVDE-NLS-TAT-His6 mice revealed survival advantages in these active treatment groups, with log-rank test p values of 0.135 and 0.037, respectively (FIG. 6A). A comparable analysis of another encapsulated enzyme compared to controls (FIG. 6B) revealed less of a survival advantage for cv-pdg-NLS-His6 (p=0.598) than was observed for the two UVDE groups.

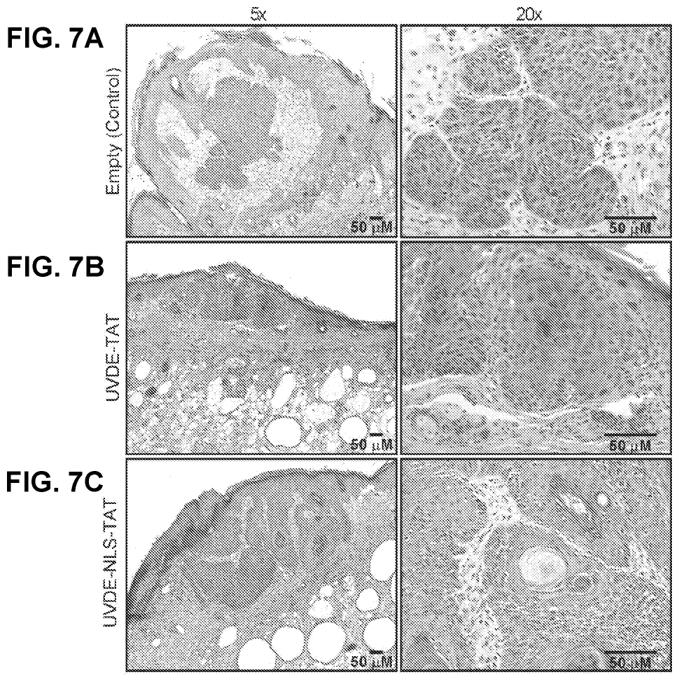

At the time when mice were euthanized, skin strips were harvested and processed for H&E staining. Even though the total tumor burden at 33 weeks and the time-to-death analyses revealed significant differences, histologic analyses of the tumors that formed were indistinguishable among the 4 groups. All examined tumors were asymmetric and poorly circumscribed epithelial neoplasms characterized by irregular aggregates extending into the superficial to deep dermis, or, in some cases, into the subcutis and underlying muscle (FIGS. 7A-7C). Atypical keratinocytes had enlarged, hyperchromatic and pleomorphic nuclei, were undergoing mitoses or apoptosis, and exhibited variable levels of eosinophilic cytoplasms. These features are highly characteristic of SCCs.

The temporal hierarchy of NER of UV-induced dipyrimidine photoproducts in mammalian cells is characterized by not only rapid recognition and excision of 6-4 PPs (which are preferentially formed in open chromatin regions), but also preferential repair of CPDs in actively transcribed genes (Spivak, Arch Toxicol 90:2583-2594, 2016; Spivak & Ganesan, DNA Repair (Amst) 19:64-70, 2014; Spivak & Hanawalt, Mutat Res 776:24-30, 2015). The remaining dipyrimidine photoproducts are repaired at greatly reduced rates that can extend over several days following a UV dose equivalent to a minimal sunburn. If cytosine bases are at either the 5′ or 3′ position or both, these damages can undergo spontaneous deamination to uracil that is highly mutagenic if replicated. CPDs remaining in the genome also function as one of the main causes of UV-induced immune suppression (Damiani & Ullrich, Prog Lipid Res 63:14-27, 2016; Hori et al., World J Transplant 5:11-18, 2015). Thus, the potential deleterious effects of unrepaired DNA damage have the capacity to increase genomic instability and reduce immune surveillance, both of which increase the risk of cellular transformation.

A strategy to mitigate these issues is to enhance DNA repair capacity in damaged cells by activating an alternative DNA repair pathway. Since less complex organisms possess alternative pathways to repair pyrimidine dimers, such as photoreactivation, BER, and NIR, the methodological challenge is to efficiently deliver sufficient levels of a repair enzyme to activate analogous alternative pathways in mammals. In the case of photoreactivation, there is not only the issue of delivering CPD- and 6-4 PP-specific photolyases, but also establishing conditions required to irradiate with sufficient amounts of appropriately tuned wavelengths of visible light. To avoid the problems associated with photoreactivation, the delivery of CPD-specific DNA glycosylase/AP lyases to initiate BER, or the introduction of UV endonucleases to activate NIR, offer feasible alternatives.

Differences in substrate specificities allow each enzyme to provide specific advantages, with pdgs recognizing only CPDs and ring-fragmented purines, and UVDEs removing both 6-4 PPs and CPDs, but not the ring-fragmented purines. The initiation of repair of both dipyrimidine photoproducts may have important biological implications. Since NEIL1- and OGG1-initiated repair of the ring-fragmented purines is likely to be sufficient to minimize the effects of these specific UVB-induced DNA lesions (Calkins et al., DNA Repair (Amst) 48:43-50, 2016), it is hypothesized that the repair of both types of dipyrimidine photoproducts leads to suppression of UVB-induced carcinogenesis.

Without being bound by any one theory, there are several potential mechanisms through which the topical delivery of UVDE could reduce the severity of UVB-induced carcinogenesis. These include: 1) increased, high-fidelity cellular DNA repair for CPDs and 6-4 PPs such that both dipyrimidine adducts are removed at an accelerated rate, thus reducing UVB-induced mutagenesis and carcinogenesis; 2) increased cell death in severely damaged cells via rapid initiation of single-strand breaks at CPDs and 6-4 PPs, thus killing potentially carcinogenic cells from undergoing error-prone replication; and 3) minimization of UV-induced immune suppression.

The mechanism of UVDE-initiated rapid repair of CPDs and 6-4 PPs, in which the repair patch is anticipated to be synthesized with high fidelity, is based on the following studies. UVDE initiates repair at both dipyrimidine photoproducts by incision immediately 5′ to the damage (Avery et al., 1999, supra; Bowman et al., Nucleic Acids Res 22:3026-3032, 1994; Takao et al., Nucleic Acids Res 24:1267-1271, 1996), followed by long-patch BER in conjunction with Rad27/Fen1, XRCC1, PARP1, and DNA polymerase (Alleva et al., Biochemistry 39:2659-2666, 2000; Asagoshi et al., DNA Repair (Amst) 9:109-119, 2010; Okano et al., J Biol Chem 275:32635-32641, 2000; Yoon et al., Biochemistry 38:4809-4817, 1999). Further, in UV-irradiated XPA cells expressing the Neurospora crassa UVDE, single-strand breaks were introduced immediately following irradiation and were efficiently repaired, resulting in enhanced survival, approaching that of wild-type cells (Asagoshi et al., 2010, infra; Okano et al., 2000, infra). It was also demonstrated via expression of UVDE in repair-proficient and -deficient S. pombe, that introduction of the enzyme resulted in the initiation of repair of CPDs in both the nucleus and mitochondria, but that nuclear-localized repair was responsible for conferring enhanced survival in NER-deficient cells (Yasuhira & Yasui, J Biol Chem 275:11824-11828, 2000). These mechanisms may occur via high fidelity reactions, since UVDE recognition of CPDs involves a quadruple flipping mechanism in which both the damaged dipyrimidine and the two complementary purines are extrahelical (Meulenbroek et al., Nucleic Acids Res 41:1363-1371, 2013; Tsutakawa et al., DNA Repair (Amst) 19:95-107, 2014).

Collectively, these studies present evidence that following UVB irradiation, UVDE-initiated repair results in rapid, long-patch processes improving survival in DNA repair-deficient cells. The final product of long-patch BER is anticipated to be error-free since the undamaged complementary strand is available for repair-patch synthesis. This rapid repair of dipyrimidine photoproducts is also expected to greatly minimize UV-induced immune suppression since one of the primary signals for initiating this suppression is the duration of unrepaired CPDs in genomic DNAs (Damiani & Ullrich, 2016, supra; Prasad & Katiyar, Photochem Photobiol 93:930-936, 2017; Strickland & Kripke, Clin Plast Surg 24:637-647, 1997; Ullrich & Byrne, J Invest Dermatol 132:896-905, 2012). This hypothesis is also consistent with data from the XP clinical trial using T4-pdg in which suppression of new tumors could at least partially be explained by enhanced immune surveillance.

Overall, these data provide compelling evidence for the benefit of topical delivery of a truncated DNA repair enzyme, UVDE, containing at least one heterologous targeting sequence at its C-terminus, to repair UV-induced DNA damage in skin of a subject exposed to UV irradiation, to reduce the total tumor burden of a subject exposed to UV irradiation, and to decrease the severity of a UV-induced inflammatory response of a subject exposed to UV irradiation. Clinical trials with compositions of the present disclosure will be conducted with XP patients and organ-transplant patients who have higher rates of NMSC.

Example 2. Increased DNA repair of UVB irradiation in Gottingen minipig model results in markers of decreased inflammatory responses.

UVB irradiation is known to induce pro-inflammatory signaling, increased white blood cell count and eventually immune suppression. These responses are induced by sufficient UVB irradiation that produces a mean erythemal dose (MED). The severity of these responses is related to the rate of DNA repair of cyclobutane pyrimidine dimers, in which increased DNA repair leads to decreases in the severity of these inflammatory responses. Thus, increased DNA repair is anticipated to decrease the severity of these responses. To provide preclinical data in a skin model that closely resembles human, pig models are preferred.

Methods. The Gottingen minipig was chosen as the animal model because it is an accepted non-rodent species for preclinical toxicity testing by regulatory agencies. Animals were assigned to groups by a stratified randomization scheme designed to achieve similar group mean body weights. Housing and care were as specified in the USDA Animal Welfare Act (9 CFR, Parts 1, 2, and 3) and as described in the Guide for the Care and Use of Laboratory Animals from the National Research Council. The DNA repair enzymes encapsulated in liposomes and control empty liposomes were administered to the appropriate animals dermally once daily from Days 1 to 4. The final dose was administered on Day 5 and the test site was rinsed following the 0 hour UVB exposure. The dose volume for each animal was a set volume of 10 mL per animal.

Prior to Day 1 (after randomization), the test site (15cm×20cm) was delineated by placing a tattoo at the corner of each test site. The UVB irradiation grid was also delineated within the test site by the same procedure. The tattoo procedure followed Testing Facility SOP-3767 with the exception that the tattoos were applied to the minipig's back. The dorsal surface was prepared by close clipping of the hair with a small animal clipper prior to the first dose and as often as necessary thereafter. Care was taken during the clipping procedure to avoid abrasion of the skin. The dosing materials were applied directly to the skin in a uniform layer over each designated area by gentle inunction with a disposable plastic applicator. A target area of 10% of the total body surface area was covered with a thin, uniform film of the appropriate dosing material. The area of application was estimated. All animals had intact skin. All 12 animals (3 each for control, cv-pdg-NLS-His6, UVDE-TAT-His6, and UVDE-NLS-TAT-His6) had a grid tattoo drawn on the back. The grid covered the entire test site. UVB irradiation was performed at 0, 2, 6, 24, and 72 hours prior to euthanasia.

A UVB bulb in an exposure housing was used to deliver the equivalent of either 1X or a 2X MED dose. A quartz glass filter was placed immediately on top of the exposed skin for the applicable test site(s). The UVB lamp was positioned immediately above the quartz glass filter. To deliver a dose rate of 2400 μW/cm², lamps were positioned immediately over the site to be irradiated with the quartz glass filter on top of the applicable site. To achieve a 1 MED (1X sunburn) dose, a 50 second exposure was required. To achieve a 2 MED (2X sunburn) dose, a 100 second exposure was required. During UVB exposure, the remainder of the skin was occluded from any unintentional UVB exposure. Energy of the UV radiation was previously measured by a calibrated UVB meter and was stable. Blood was collected by venipuncture of the vena cava.

All animals had two 2 cm×2 cm biopsy skin samples collected from each quadrant following euthanasia. One sample from each quadrant was collected, placed in a cryovial, frozen in liquid nitrogen, and frozen in a freezer set to maintain at −70° C. for immunohistochemical analyses.

### Results.

Hematology. As described above, four groups of mini-pigs were administered both a 1 and 2 MED dose to a total 10% of total skin surface. At the time of euthanasia, white blood cell counts were evaluated in the DNA repair enzyme-treated liposomes versus pigs administered the control. Relative to the control values, circulating lymphocytes decreased 10, 17, and 31% for cv-pdg-NLS-His6, UVDE-TAT-His6, and UVDE-NLS-TAT-His6, respectively. Relative to the control values, circulating monocytes decreased 3, 2, and 23% for cv-pdg-NLS-His6, UVDE-TAT-His6, and UVDE-NLS-TAT-His6, respectively. Relative to the control values, circulating eosinophils increased 29% for cv-pdg-NLS-His6, but decreased 31 and 50% for UVDE-TAT-His6, and UVDE-NLS-TAT-His6, respectively. Analyses of circulating red blood cells were unchanged across all groups and these data served as an excellent control for the changes in white blood cell count in the cells. Additionally, increased circulating ketone and protein levels were elevated in the control group versus any of the 3 enzyme-treated groups.

These data show that delivery of DNA repair enzymes decreases the overall inflammatory reaction to UVB irradiation, and suggest that the most profound effect is created by the topical delivery of UVDE-NLS-TAT-His6, followed by UVDE-TAT-His6, followed by cv-pdg-NLS-His6.

As will be understood by one of ordinary skill in the art, each embodiment disclosed herein can include, consist essentially of or consist of its particular stated element, step, ingredient or component. Thus, the terms “include” or “including” should be interpreted to recite: “include, consist of, or consist essentially of.” As used herein, the transition term “include” or “includes” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in an embodiment's ability to provide a statistically significant beneficial effect in repair of DNA damage; decreasing tumor size, number of tumors, and/or total tumor burden caused by UV irradiation; decreasing risk of death (hazard ratios) and/or increasing survival of subjects exposed to UV irradiation; decreasing UV-induced immune suppression; decreasing frequency of UV-induced nonmelanoma skin cancers (NMSCs).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A recombinant polypeptide comprising:
a truncated UV damage endonuclease (UVDE) shown in amino acids 229 to 599 of SEQ ID NO: 16, and
a transactivator of transcription (TAT) amino acid sequence of SEQ ID NO: 2 at the carboxy-terminus (C-terminus) of the UVDE.

2. A recombinant polypeptide comprising:
a truncated UV damage endonuclease (UVDE), wherein the truncation is amino-terminal (N-terminal) to a conserved region of the UVDE required for enzymatic activity, and
at least one heterologous targeting sequence at the carboxy-terminus (C-terminus) of the UVDE.

3. (canceled)

4. The recombinant polypeptide of claim 1, wherein the truncated UVDE is Schizosaccharomyces pompe Uve1p lacking the first 228 amino acids of SEQ ID NO: 16.

5. The recombinant polypeptide of claim 2, wherein the at least one heterologous targeting sequence comprises:
a cell penetrating peptide; or
a nuclear localization signal (NLS); or
a NLS and a TAT protein transduction domain.

6. The recombinant polypeptide of claim 2, wherein the recombinant polypeptide comprises amino acids 1-383 of SEQ ID NO: 12 or amino acids 1-391 of SEQ ID NO: 14.

7. The recombinant polypeptide of claim 2, wherein the recombinant polypeptide further comprises one or more purification tags.

8. The recombinant polypeptide of claim 2, wherein the recombinant polypeptide further comprises at least one sequence that is specifically recognized by a protease, and which protease-recognition sequence is positioned between the truncated UVDE and the at least one heterologous targeting sequence.

9. The recombinant polypeptide of claim 2, wherein the recombinant polypeptide is encapsulated in a liposome.

10. A recombinant polynucleotide encoding:
a truncated UV damage endonuclease (UVDE) sequence, wherein the truncation is 5′ to a conserved region of the UVDE sequence required for enzymatic activity; and
at least one heterologous targeting sequence at the 3′ end of the UVDE sequence.

11. (canceled)

12. The recombinant polynucleotide of claim 10, wherein the truncated UVDE sequence is Schizosaccharomyces pombe Uve1 lacking the first 684 nucleotides of SEQ ID NO: 15.

13. The recombinant polynucleotide of claim 10, wherein the at least one heterologous targeting sequence comprises:
a cell penetrating peptide sequence;
a nuclear localization signal (NLS) sequence; or
a NLS sequence and a TAT cell penetrating peptide sequence.

14. The recombinant polynucleotide of claim 10, wherein the recombinant polynucleotide comprises nucleotides 1-1149 of SEQ ID NO: 11 or nucleotides 1-1173 of SEQ ID NO: 13.

15. The recombinant polynucleotide of claim 10, wherein the recombinant polynucleotide further comprises one or more purification tag sequences.

16. The recombinant polynucleotide of claim 10, wherein the recombinant polynucleotide further comprises at least one sequence that encodes a peptide sequence specifically recognized by a protease, and which protease-recognition sequence encoding sequence is positioned between the sequence encoding the truncated UVDE and the sequence encoding the at least one heterologous targeting sequence.

17. A vector comprising the recombinant polynucleotide of claim 10.

18. A host cell comprising the recombinant polypeptide of claim 2 or a recombinant polynucleotide encoding that polypeptide.

19. A topical formulation comprising a therapeutically effective amount of the recombinant polypeptide of claim 2, or a recombinant polynucleotide encoding that polypeptide.

20. The topical formulation of claim 19, wherein the topical formulation is incorporated in a lotion, a cream, an ointment, a paste, a powder, a sunscreen, a liquid, an aerosol, a suspension, an emulsion, a foam, a gel, a hydrogel, a plaster, a patch, a bandage, a wipe, a microsponge, an elastomer, or a film.

21. The topical formulation of claim 19, wherein the recombinantpolypeptide or the recombinant polynucleotide is encapsulated in a liposome.

22. A composition comprising the recombinant polypeptide of claim 2, or a recombinant polynucleotide encoding that polypeptide, and a pharmaceutically acceptable carrier.

23. The composition of claim 22, wherein the recombinant polypeptide or the recombinant polynucleotide is encapsulated in a liposome.

24. A method comprising contacting skin of a subject with a therapeutically effective amount of a composition comprising the recombinant polypeptide of claim 2, or a recombinant polynucleotide encoding that polypeptide.

25. The method of claim 24, which is a method for:
repairing UV-induced DNA damage in skin of a subject;
reducing the number of tumors, size of tumors and/or total tumor burden in a subject exposed to UV irradiation; and/or
treating or reducing the risk of a skin disorder in a subject.

26. The method of claim 25, wherein the skin disorder is melanoma, non-melanoma skin cancer (NMSC), actinic keratosis (AK), angiofibroma, pachyonychia congenita, or xeroderma pigmentosum.

27. The method of claim 26, wherein the NMSC comprises basal cell carcinoma (BCC), squamous cell carcinoma (SCC), Merkel cell carcinoma, cutaneous (skin) lymphoma, Kaposi sarcoma, skin adnexal tumors and sarcomas.

28. The method of claim 27, wherein the subject has xeroderma pigmentosum or is an organ-transplant patient.

29. The method of claim 28, wherein the frequency of AK is reduced.

30. A method for treating or reducing UV-induced immunosuppression in a subject in need thereof comprising administering a therapeutically effective amount of the recombinant polypeptide of claim 2, or a recombinant polynucleotide encoding that polypeptide, to the subject to treat or reduce UV-induced immunosuppression in the subject as compared to a subject in need thereof not administered a therapeutically effective amount of the recombinant polypeptide or the recombinant polynucleotide.

31. The method of claim 30, wherein an increase in repair of CPDs and/or 6-4 PPs occurs in the subject administered the therapeutically effective amount of the recombinant polypeptide or the recombinant polynucleotide as compared to the subject not administered the therapeutically effective amount of the recombinant polypeptide or the recombinant polynucleotide.

32. A method for decreasing severity of a UV-induced inflammatory response in a subject in need thereof comprising administering a therapeutically effective amount of the recombinant polypeptide of claim 2, or a recombinant polynucleotide encoding that polypeptide, to the subject to decrease the severity of the UV-induced inflammatory response in the subject as compared to a subject in need thereof not administered a therapeutically effective amount of the recombinant polypeptide or the recombinant polynucleotide.

33. The method of claim 32, wherein the number of circulating lymphocytes, monocytes and/or eosinophils is reduced in the subject administered the therapeutically effective amount of the recombinant polypeptide or the recombinant polynucleotide.


SEQUENCE LISTING (85 sequences)

SEQ ID NO: 1 - DNA, 33 bp, HIV
tatggccgcaaaaagcgccgtcagcgccgtcgc

SEQ ID NO: 2 - protein, 11 aa, HIV
YGRKKRRQRRR

SEQ ID NO: 3 - DNA, 57 bp, Artificial Sequence
  Nucleotide sequence encoding a nuclear localization signal (NLS) and TAT
ccaaagaagaggaaaaggaggctatatggccgcaaaaagcgccgtcagcgccgtcgc

SEQ ID NO: 4 - protein, 19 aa, Artificial Sequence
  Amino acid sequence for an NLS-TAT
PKKRKRRLYGRKKRRQRRR

SEQ ID NO: 5 - DNA, 21 bp, Artificial Sequence
  Nucleotide sequence encoding a nuclear localization signal (NLS) used in the UVDE-NLS-TAT-His6 expression construct
ccaaagaagaggaaaaggagg

SEQ ID NO: 6 - protein, 7 aa, Artificial Sequence
  Amino acid sequence of NLS used in the UVDE-NLS-TAT-His6 expression construct
PKKRKRR

SEQ ID NO: 7 - protein, 6 aa, Artificial Sequence
  Amino acid sequence of a linker
LAAALE

SEQ ID NO: 8 - protein, 6 aa, Artificial Sequence
  hexa-histidine tag
HHHHHH

SEQ ID NO: 9 - DNA, 30 bp, Artificial Sequence
  Nucleotide sequence encoding a nuclear localization signal (NLS) used in the cv-pdg-NLS-His6 expression construct
cccgggccaaagaaaaagaggaagaggcta

SEQ ID NO: 10 - protein, 10 aa, Artificial Sequence
  Amino acid sequence for an NLS used in the cv-pdg-NLS-His6 expression construct
PGPKKKRGRL

SEQ ID NO: 11 - DNA, 1167 bp, Artificial Sequence
  Nucleotide sequence encoding UVDE-TAT-His6
gatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaagctttatggccgcaaaaagcgccgtcagcgccgtcgccaccaccaccaccaccac

SEQ ID NO: 12 - protein, 389 aa, Artificial Sequence
  Amino acid sequence for a UVDE-TAT-His6 recombinant polypeptide
DDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEKLYGRKKRRQRRRHHHHHH

SEQ ID NO: 13 - DNA, 1191 bp, Artificial Sequence
  Nucleotide sequence encoding UVDE-NLS-TAT-His6
gatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaagcttccaaagaagaggaaaaggaggctatatggccgcaaaaagcgccgtcagcgccgtcgccaccaccaccaccaccac

SEQ ID NO: 14 - protein, 397 aa, Artificial Sequence
  Amino acid sequence for a UVDE-NLS-TAT-His6 recombinant polypeptide
DDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEKLPKKRKRRLYGRKKRRQRRRHHHHHH

SEQ ID NO: 15 - DNA, 1800 bp, Schizosaccharomyces pombe
atgcttaggctattgaaacgaaatattcaaatctctaaacgcattgttttcaccatattaaaacaaaaggcatttaaaggtaatcatccttgtgtaccgtcggtttgtaccattacttactctcgttttcattgtttacccgatacccttaaaagtttacttccaatgagctcaaaaaccacactctcaatgttaccgcaagttaatatcggtgcgaattcattctctgccgaaacaccagtcgacttaaaaaaagaaaatgagactgagttagctaatatcagtggacctcacaaaaaaagtacttctacgtctacacgaaagagggcacgtagcagtaaaaagaaagcgacagattctgtttccgataaaattgatgagtctgttgcgtcctatgattcttcaactcatcttaggcgatcgtcgagatcaaaaaaaccggtcaactacaattcctcgtcagaatccgaatcggaggagcaaattagtaaagctactaaaaaagttaaacaaaaagaggaagaggagtatgttgaagaagtcgacgaaaagtctcttaaaaatgaaagtagctctgacgagttcgaaccggttgtgccggaacagttggaaactccaatttctaaacgaagacggtctcgttcttctgcaaaaaatttagaaaaagaatctacaatgaatcttgatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaaataa

SEQ ID NO: 16 - protein, 599 aa, Schizosaccharomyces pombe
MLRLLKRNIQISKRIVFTILKQKAFKGNHPCVPSVCTITYSRFHCLPDTLKSLLPMSSKTTLSMLPQVNIGANSFSAETPVDLKKENETELANISGPHKKSTSTSTRKRARSSKKKATDSVSDKIDESVASYDSSTHLRRSSRSKKPVNYNSSSESESEEQISKATKKVKQKEEEEYVEEVDEKSLKNESSSDEFEPVVPEQLETPISKRRRSRSSAKNLEKESTMNLDDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEK

SEQ ID NO: 17 - protein, 7 aa, Artificial Sequence
  Amino acid sequence for an SV40 large T antigen nuclear localization signal (NLS)
PKKKRKV

SEQ ID NO: 18 - protein, 9 aa, Artificial sequence
  Amino acid sequence for a c-Myc nuclear localization signal (NLS)
PAAKRVKLD

SEQ ID NO: 19 - protein, 19 aa, Artificial Sequence
  Amino acid sequence for a putative consensus sequence of the bipartite nuclear localization signal (NLS) (K/R)(K/R)X10-12(K/R)3/5
  MISC_FEATURE (1)..(1): K or R
  MISC_FEATURE (2)..(2): K or R
  MISC_FEATURE (3)..(3): Positions 3 to 12/3 to 14, any amino acid or none (such that there are 10-12 amino acids)
  MISC_FEATURE (4)..(4): Positions 3 to 12/3 to 14, any amino acid or none (such that there are 10-12 amino acids)
  MISC_FEATURE (5)..(5): Positions 3 to 12/3 to 14, any amino acid or none (such that there are 10-12 amino acids)
  MISC_FEATURE (6)..(6): Positions 3 to 12/3 to 14, any amino acid or none (such that there are 10-12 amino acids)
  MISC_FEATURE (7)..(7): Positions 3 to 12/3 to 14, any amino acid or none (such that there are 10-12 amino acids)
  MISC_FEATURE (8)..(8): Positions 3 to 12/3 to 14, any amino acid or none (such that there are 10-12 amino acids)
  MISC_FEATURE (9)..(9): Positions 3 to 12/3 to 14, any amino acid or none (such that there are 10-12 amino acids)
  MISC_FEATURE (10)..(10): Positions 3 to 12/3 to 14, any amino acid or none (such that there are 10-12 amino acids)
  MISC_FEATURE (11)..(11): Positions 3 to 12/3 to 14, any amino acid or none (such that there are 10-12 amino acids)
  MISC_FEATURE (12)..(12): Positions 3 to 12/3 to 14, any amino acid or none (such that there are 10-12 amino acids)
  MISC_FEATURE (13)..(13): Positions 3 to 12/3 to 14, any amino acid or none (such that there are 10-12 amino acids)
  MISC_FEATURE (14)..(14): Positions 3 to 12/3 to 14, any amino acid or none (such that there are 10-12 amino acids)
  MISC_FEATURE (15)..(15): Positions 15 to 19, any amino acid so long as 3 of the 5 are either K or R
  MISC_FEATURE (16)..(16): Positions 15 to 19, any amino acid so long as 3 of the 5 are either K or R
  MISC_FEATURE (17)..(17): Positions 15 to 19, any amino acid so long as 3 of the 5 are either K or R
  MISC_FEATURE (18)..(18): Positions 15 to 19, any amino acid so long as 3 of the 5 are either K or R
  MISC_FEATURE (19)..(19): Positions 15 to 19, any amino acid so long as 3 of the 5 are either K or R
XXXXXXXXXXXXXXXXXXX

SEQ ID NO: 20 - protein, 7 aa, Artificial Sequence
  exemplary nuclear localization signal (NLS) sequence
PKKKRMV

SEQ ID NO: 21 - protein, 10 aa, Artificial Sequence
  exemplary nuclear localization signal (NLS) sequence
PKKKRKVEDP

SEQ ID NO: 22 - protein, 8 aa, Artificial Sequence
  exemplary nuclear localization signal (NLS) sequence
PKKGSKKA

SEQ ID NO: 23 - protein, 7 aa, Artificial Sequence
  exemplary nuclear localization signal (NLS) sequence
PKTKRKV

SEQ ID NO: 24 - protein, 11 aa, Artificial Sequence
  exemplary nuclear localization signal (NLS) sequence
CGGPKKKRKVG

SEQ ID NO: 25 - protein, 7 aa, Artificial Sequence
  exemplary nuclear localization signal (NLS) sequence
PKKKIKV

SEQ ID NO: 26 - protein, 34 aa, Artificial Sequence
  exemplary nuclear localization signal (NLS) sequence
CYDDEATADSQHSTPPKKKRKVEDPKDFESELLS

SEQ ID NO: 27 - protein, 13 aa, Artificial Sequence
  exemplary nuclear localization signal (NLS) sequence
CGYGPKKKRKVGG

SEQ ID NO: 28 - protein, 13 aa, Artificial Sequence
  exemplary nuclear localization signal (NLS) sequence
CGYGVSRKRPRPG

SEQ ID NO: 29 - protein, 8 aa, Artificial Sequence
  exemplary nuclear localization signal (NLS) sequence
APTKRKGS

SEQ ID NO: 30 - protein, 11 aa, Artificial Sequence
  exemplary nuclear localization signal (NLS) sequence
APKRKSGVSKC

SEQ ID NO: 31 - protein, 7 aa, Artificial Sequence
  exemplary nuclear localization signal (NLS) sequence
PNKKKRK

SEQ ID NO: 32 - protein, 12 aa, Artificial Sequence
  exemplary nuclear localization signal (NLS) sequence
EEDGPQKKKRRL

SEQ ID NO: 33 - protein, 7 aa, Artificial Sequence
  exemplary nuclear localization signal (NLS) sequence
GKKRSKA

SEQ ID NO: 34 - protein, 11 aa, Artificial Sequence
  exemplary nuclear localization signal (NLS) sequence
CGGLSSKRPRP

SEQ ID NO: 35 - protein, 12 aa, Artificial Sequence
  exemplary nuclear localization signal (NLS) sequence
LKDKDAKKSKQE

SEQ ID NO: 36 - protein, 10 aa, Artificial Sequence
  exemplary nuclear localization signal (NLS) sequence
GNKAKRQRST

SEQ ID NO: 37 - protein, 11 aa, Artificial Sequence
  exemplary nuclear localization signal (NLS) sequence
PFLDRLRRDQK

SEQ ID NO: 38 - protein, 9 aa, Artificial Sequence
  exemplary nuclear localization signal (NLS) sequence
SVTKKRKLE

SEQ ID NO: 39 - protein, 9 aa, Artificial Sequence
  exemplary nuclear localization signal (NLS) sequence
SASKRRRLE

SEQ ID NO: 40 - protein, 11 aa, Artificial Sequence
  exemplary nuclear localization signal (NLS) sequence
PPKKRMRRRIE

SEQ ID NO: 41 - protein, 18 aa, Artificial Sequence
  exemplary nuclear localization signal (NLS) sequence
YRKCLQAGMNLEARKTKK

SEQ ID NO: 42 - protein, 10 aa, Artificial Sequence
  exemplary nuclear localization signal (NLS) sequence
KIKGIQQATA

SEQ ID NO: 43 - protein, 17 aa, Artificial Sequence
  exemplary nuclear localization signal (NLS) sequence
RKDRRGGRMLKHKRQRD

SEQ ID NO: 44 - protein, 36 aa, Artificial Sequence
  exemplary nuclear localization signal (NLS) sequence
DGEGRGEVGSAGDMRAMINACIDNLWPSPLMIKRSK

SEQ ID NO: 45 - protein, 8 aa, Artificial Sequence
  exemplary nuclear localization signal (NLS) sequence
RKFKKFNK

SEQ ID NO: 46 - protein, 8 aa, Artificial Sequence
  exemplary nuclear localization signal (NLS) sequence
PLLKKIKQ

SEQ ID NO: 47 - protein, 8 aa, Artificial Sequence
  exemplary nuclear localization signal (NLS) sequence
PPQKKIKS

SEQ ID NO: 48 - protein, 7 aa, Artificial Sequence
  exemplary nuclear localization signal (NLS) sequence
PQPKKKP

SEQ ID NO: 49 - protein, 9 aa, Artificial Sequence
  exemplary nuclear localization signal (NLS) sequence
SKRVAKRKL

SEQ ID NO: 50 - protein, 14 aa, Artificial Sequence
  exemplary nuclear localization signal (NLS) sequence
MTGSKTRKHRGSGA

SEQ ID NO: 51 - protein, 6 aa, Artificial Sequence
  exemplary nuclear localization signal (NLS) sequence
RHRKHP

SEQ ID NO: 52 - protein, 6 aa, Artificial Sequence
  exemplary nuclear localization signal (NLS) sequence
KRRKHP

SEQ ID NO: 53 - protein, 6 aa, Artificial Sequence
  exemplary nuclear localization signal (NLS) sequence
KYRKHP

SEQ ID NO: 54 - protein, 6 aa, Artificial Sequence
  exemplary nuclear localization signal (NLS) sequence
KHRRHP

SEQ ID NO: 55 - protein, 6 aa, Artificial Sequence
  exemplary nuclear localization signal (NLS) sequence
KHKKHP

SEQ ID NO: 56 - protein, 6 aa, Artificial Sequence
  exemplary nuclear localization signal (NLS) sequence
RHLKHP

SEQ ID NO: 57 - protein, 17 aa, Artificial Sequence
  exemplary nuclear localization signal (NLS) sequence
PETTWRRRGRSPRRRTP

SEQ ID NO: 58 - protein, 15 aa, Artificial Sequence
  exemplary nuclear localization signal (NLS) sequence
SPRRRRSPRRRRSQS

SEQ ID NO: 59 - protein, 9 aa, Artificial Sequence
  exemplary nuclear localization signal (NLS) sequence
ASKSRKRKL

SEQ ID NO: 60 - protein, 16 aa, Artificial Sequence
  exemplary nuclear localization signal (NLS) sequence
GGLCSARLHRHALLAT

SEQ ID NO: 61 - protein, 18 aa, Artificial Sequence
  exemplary nuclear localization signal (NLS) sequence
DTREKKKFLKRRLLRLDE

SEQ ID NO: 62 - DNA, 18 bp, Artificial Sequence
  sequence encoding a hexahistadine tag
caccaccaccaccaccac

SEQ ID NO: 63 - DNA, 1113 bp, Artificial Sequence
  Nucleotide sequence encoding a truncated Schizosaccharomyces pombe UVDE lacking amino acid residues 1 - 228
gatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaag

SEQ ID NO: 64 - protein, 371 aa, Artificial Sequence
  ?228-UVDE Amino acid sequence for a truncated Schizosaccharomyces pombe UVDE lacking amino acid residues 1 - 228
DDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEK

SEQ ID NO: 65 - protein, 5 aa, Artificial Sequence
  Amino acid sequence of a linker
AAALE

SEQ ID NO: 66 - protein, 18 aa, Artificial Sequence
  Exemplary cell-penetrating peptide
KLALKLALKALKAALKLA

SEQ ID NO: 67 - protein, 34 aa, Artificial Sequence
  Exemplary cell-penetrating peptide
DAATATRGRSAASRPTERPRAPARSASRPRRPVE

SEQ ID NO: 68 - protein, 11 aa, Artificial Sequence
  Exemplary cell-penetrating peptide
YARVRRRGPRR

SEQ ID NO: 69 - protein, 23 aa, Artificial Sequence
  Exemplary cell-penetrating peptide
TKRRITPKDVIDVRSTVTTEINT

SEQ ID NO: 70 - protein, 16 aa, Artificial Sequence
  Exemplary cell-penetrating peptide
AAVALLPAVLLALLAP

SEQ ID NO: 71 - protein, 22 aa, Artificial Sequence
  Exemplary cell-penetrating peptide
MVTVLFRRLRIRRACGPPRVRV

SEQ ID NO: 72 - protein, 24 aa, Artificial Sequence
  Exemplary cell-penetrating peptide
GALFLGWLGAAGSTMGAPKKKRKV

SEQ ID NO: 73 - protein, 16 aa, Artificial Sequence
  Exemplary cell-penetrating peptide
RQIKIWFQNRRMKWKK

SEQ ID NO: 74 - protein, 21 aa, Artificial Sequence
  Exemplary cell-penetrating peptide
KETWWETWWTEWSQPKKKRKV

SEQ ID NO: 75 - protein, 17 aa, Artificial Sequence
  Exemplary cell-penetrating peptide
LLILRRRIRKQAHAHSK

SEQ ID NO: 76 - protein, 27 aa, Artificial Sequence
  Exemplary cell-penetrating peptide
GALFLGFLGAAGSTMGAWSQPKKKRKV

SEQ ID NO: 77 - protein, 9 aa, Artificial Sequence
  Exemplary cell-penetrating peptide
RRWWRRWRR

SEQ ID NO: 78 - protein, 18 aa, Artificial Sequence
  Exemplary cell-penetrating peptide
VRLPPPVRLPPPVRLPPP

SEQ ID NO: 79 - protein, 18 aa, Artificial Sequence
  Exemplary cell-penetrating peptide
RGGRLSYSRRRFSTSTGR

SEQ ID NO: 80 - protein, 10 aa, Artificial Sequence
  Exemplary cell-penetrating peptide
RRLSYSRRRF

SEQ ID NO: 81 - protein, 13 aa, Artificial Sequence
  Exemplary cell-penetrating peptide
GRKKRRQRRRPPQ

SEQ ID NO: 82 - protein, 21 aa, Artificial Sequence
  Exemplary cell-penetrating peptide
AGYLLGKINLKALAALAKKIL

SEQ ID NO: 83 - protein, 26 aa, Artificial Sequence
  Exemplary cell-penetrating peptide
GWTLNSAGYLLKINLKALAALAKKIL

SEQ ID NO: 84 - protein, 490 aa, Artificial Sequence
  Amino Acid sequence of His6SUMO-UVDE-TAT
MHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGDDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEKLYGRKKRRQRRR

SEQ ID NO: 85 - DNA, 1473 bp, Artificial Sequence
  Nucleic acid sequence of His6SUMO-UVDE-TAT
atgcatcatcaccaccatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcaccgcgaacagattggaggtgatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaaactatatggccgcaaaaagcgccgtcagcgccgtcgctaa